(19) United States
(12) Patent Application Publication
(10) Pub. No.: US 2024/0122842 A1
(43) Pub. Date: Apr. 18, 2024

(54) SYSTEM, PRODUCT AND METHOD FOR MAINTAINING THE VAGINAL MICROBIOME

(71) Applicant: VAGINAL BIOME SCIENCE, INC., Eugene, OR (US)

(72) Inventor: Wendy STRGAR, Eugene, OR (US)

(21) Appl. No.: 18/539,018

(22) Filed: Dec. 13, 2023

Related U.S. Application Data
(62) Division of application No. 18/048,761, filed on Oct. 21, 2022.
(60) Provisional application No. 63/271,021, filed on Oct. 22, 2021.

(51) Int. Cl. A61K 8/99; A61K 8/02; A61K 8/19; A61K 8/365; A61K 8/42; A61K 8/46; A61K 8/73; A61K 8/9794; A61Q 19/10

(52) U.S. Cl. CPC A61K 8/99; A61K 8/0208; A61K 8/19; A61K 8/365; A61K 8/42; A61K 8/466; A61K 8/731; A61K 8/735; A61K 8/9794; A61Q 19/10

(57) ABSTRACT

A feminine hygiene system contains one or more feminine hygiene products that establish, restore, or maintain an optimal vaginal microbiome, an optimal interior environment of the vagina, and healthy vaginal tissue, including ion concentrations and ratios in vaginal secretions. A method using the feminine hygiene system establishes, restores, or maintains an optimal vaginal microbiome, an optimal interior environment of the vagina, and healthy vaginal tissue, and alleviates vaginal discomfort or symptoms arising from medical or physiological causes.

### FIELD

This document relates to materials that are applied topically to the vagina to enhance vaginal health.

### INTRODUCTION

Portions of a human body host microbial populations which support health of the tissues of the body. Together, the microbes and their environment—the tissue in which they live—constitute a “microbiome”. Healthy microbiomes provide benefits to the body and/or protection against pathogenic organisms. A microbiome may contain multiple microbes. For example, the gut microbiome hosts a variety of different microbes; some of these produce vitamins for the body, while others prevent overgrowth of yeast or pathogenic bacteria. In another example, the microbiome of the vagina hosts a variety of different microbes, especially Lactobacillus spp., which can beneficially modulate the person's immune system, and also protect against infectious organisms.

Support or supplementation of colonies of beneficial bacteria is a strategy often used by people to support healthy microbiomes. For example, yogurt or probiotic supplements may be ingested to support a healthy gut microbiome. In another example, a probiotic vaginal suppository may be inserted to support a healthy vaginal microbiome.

Prevention of growth of pathogenic bacteria through use of personal care products is a gold standard in personal hygiene. For example, using antibacterial soap during hand washing may reduce levels of pathogenic organisms growing on the skin of the hands. In another example, using a product containing appropriate levels of lactic acid may prevent growth of pathogenic bacteria in the vagina.

However, existing over-the-counter (OTC) personal care products do not promote an optimal vaginal microbiome and are therefore unable to prevent recurrence of dysbioses (states of having unhealthy microbiomes) such as bacterial vaginosis (BV). Currently, there are no products available which are able to determine common vaginal conditions or provide options to relieve symptoms and restore a healthy microbiome. In addition, some vaginal care products such as lubricants are damaging to vaginal tissue and/or the microbiome. For example, most pre-existing lubricants are formulated with an osmolality much greater than the osmolality of typical vaginal cells or bacterial cells. Using such a personal lubricant may result in the cells of the vagina releasing fluid to dilute the personal lubricant, which may result in death of the cells, damage to the vagina, and/or subsequent dryness of the vagina. It may also result in death of beneficial bacteria.

A healthy vaginal microbiome is dominated by several species of Lactobacillus, a genus of Gram-positive facultative anaerobic rod-shaped bacteria which produce lactic acid as a byproduct of metabolizing sugars. The lactic acid produced by these bacteria results in vaginal fluids having a very acidic (low) pH, in the range of 3.5 to 4.2 or 3.5 to 4.5 depending on the exact Lactobacillus species; this is one of the lowest pH levels found in the human body. This acidic environment prevents overgrowth of pathogenic bacteria and promotes health of vaginal and other reproductive tissues.

In addition to optimal pH and lactic acid levels, a healthy vaginal environment supporting a healthy vaginal microbiome must have salts (ion), sugars, and other osmotically active solutes present in the correct amounts to be osmotically balanced. Lubricants and personal care products which are hypertonic disrupt the vaginal environment, leading to increased rates of vaginal epithelial cell death and cell sloughing from the vaginal epithelium, and an increased susceptibility to dysbiosis and infection.

Health of the vagina is determined by measuring levels of Lactobacillus, pH, lactic acid levels, moisture levels, vaginal epithelial cell vitality, and levels and ratios of ions, sugars, or other osmotically active solutes. Products which are bio-matched to these parameters support healthy microbiomes and healthy vaginal tissues.

Moreover, pre-existing vaginal products commonly include other various ingredients which are harmful to the vagina. For example, these vaginal products typically include detergents and surface-active agents (surfactants), glycerol (glycerin) and other humectant/solvent excipients, and/or preservatives which typically include chlorhexidine and/or ethylenediaminetetraacetic acid (EDTA), among others. Detergents and surface-active ingredients are harmful because they are markedly toxic to mucosal epithelia, including that of the vagina. Such detergents and surface-active ingredients may include nonoxynol-9 (N9) and similar detergents, and glycerol monolaurate (GML). Glycerol (glycerin) and other humectant/solvent excipients are harmful because they increase vaginal susceptibility to disease. For example, Moench et al. (BMC Infectious Diseases 2010, 10:331) reported that the following excipients markedly increased susceptibility to herpes simplex virus (HSV-2) after a single exposure: 5% glycerol monolaurate (GML) formulated in K-Y® Warming Jelly, 5% GML as a colloidal suspension in phosphate buffered saline, K-Y® Warming Jelly alone, and both of its humectant/solvent ingredients (neat propylene glycol and neat polyethylene glycol (PEG-8).

Sodium, potassium, and calcium are important salts (ions) found in vaginal fluid that are often disrupted by pre-existing products on the market and which may also be disrupted in unhealthy vaginas. Increased sodium concentration in vaginal fluid is indicative of women with conditions resulting in loss of epithelial cell vitality, wherein epithelial cell vitality means a vaginal epithelium consisting of 30-50 layers of cells, with intact intercellular junctions, production of high quantities of glycogen, and a high transepithelial electrical resistance (TEER). For example, inflammatory conditions, use of irritating personal products, stress, hormonal changes, menstrual cycle status, radiation treatment, and some drugs may cause elevated sodium concentrations and a different sodium to potassium ratio from the normal ranges. Regarding potassium concentration, the concentration of potassium in vaginal fluid is dependent upon the flow of lubricating fluid through the epithelial surface from the underlying tissues. Therefore, women with conditions or behaviors resulting in reduced production of lubricating fluid (e.g., use of steroidal medications or other drugs; side effects of cancer treatment such as pelvic irradiation or chemotherapy; inflammatory conditions such as lichen sclerosis, lichen planus, atrophic vaginitis, desquamative inflammatory vaginitis, genital graft-vs-host disease, pelvic inflammatory disease, interstitial cystitis, Sjögren syndrome, and contact, allergic, or other dermatitis; low hormone levels due to menopause, breastfeeding, stress, pituitary or ovarian defects, pharmacological hormone suppression, or ovariectomy; cigarette smoking; excessive exercise; and depression) would exhibit decreased potassium concentrations and a different sodium to potassium ratio.

Determining the ratio of ions in vaginal fluid is used as an indication of the condition and/or health of the vaginal epithelium. Once the ratio is determined, a product suited to rectifying the out of range ion concentration, or ratio of ions, in particular sodium to potassium ratio, is determined. Assessing the ratios of ions is helpful in determining any underlying conditions or disease.

Supporting a healthy vaginal microbiome requires not only providing healthy vaginal Lactobacillus species; it also requires creating a micro-environment in which they can thrive. In the vagina, a healthy environment has a pH in the range of 3.5-4.5, fluids with osmolality in the range of 200 to 450 mOsm/kg, and salts at the appropriate concentrations and ratios.

### SUMMARY

A feminine hygiene system contains one or more products that establish, restore, or maintain an optimal vaginal microbiome including healthy vaginal Lactobacillus species, an optimal interior environment of the vagina, and healthy vaginal tissue, including, but not limited to, pH, osmolality, and ion concentrations and ratios in vaginal secretions. The system may also contain one or more assessment tools.

An assessment of various indicators in the vaginal environment may be performed as part of a subject's health profile. The assessment may include measuring one or more of the concentrations or amounts of sodium, potassium, chloride, calcium, magnesium, manganese, or other ions; ratios of sodium to potassium, sodium to calcium, sodium to chloride, sodium to magnesium, sodium to manganese, and other ion ratios; concentrations or amounts of glycogen, glucose, other monosaccharides, disaccharides, oligosaccharides or polysaccharides; moisture levels and/or mucus quantities and consistency; concentration or amount of lipopolysaccharides (LPS); presence, concentrations, or numbers of bacteria or fungi, including, but not limited to, Lactobacillus acidophilus, Lactobacillus crispatus, Lactobacillus fornicalis, Lactobacillus gallinarum, Lactobacillus gasseri, Lactobacillus helveticus, Lactobacillus iners, Lactobacillus intestinalis, Lactobacillus jensenii, Lactobacillus johnsonii, Lactobacillus ultunensis, Lacticaseibacillus casei, Lacticaseibacillus paracasei, Lacticaseibacillus rhamnosus, Lactiplantibacillus plantarum, Lentilactobacillus buchneri, Lentilactobacillus farraginis, Lentilactobacillus hilgardii, Lentilactobacillus kefiri, Lentilactobacillus parabuchneri, Levilactobacillus brevis, Levilactobacillus hammesii, Levilactobacillus parabrevis, Ligilactobacillus agilis, Ligilactobacillus hayakitensis, Ligilactobacillus ruminis, Ligilactobacillus salivarius, Limosilactobacillus antri, Limosilactobacillus fermentum, Limosilactobacillus frumenti, Limosilactobacillus gastricus, Limosilactobacillus ingluviei, Limosilactobacillus mucosae, Limosilactobacillus oris, Limosilactobacillus pontis, Limosilactobacillus reuteri, Limosilactobacillus secaliphilus, Limosilactobacillus vaginalis, Enterococcus faecalis, Gardnerella vaginalis, Chlamydia trachomatis, Neisseria gonorrhea, Ureaplasma urealyticum, Prevotella spp., Mobiluncus spp., Bacteroides spp., Peptostreptococcus spp., Fusobacterium spp., Veillonella spp., Eubacterium spp., Bifidobacterium spp., Prevotella spp., Finegoldia spp., Corynebacterium spp., Propionibacterium spp., Peptoniphilus spp., Staphylococcus spp., Anaerococcus spp., Mycoplasma hominis, Streptococcus viridans, Atopobium vaginae, Candida albicans, other Candida spp., or other species of bacteria or fungi; concentrations or amounts of D-lactic acid, L-lactic acid, or total DL-(racemic) lactic acid; pH values and ranges of vaginal fluid; integrity of the vaginal epithelium; cytokine presence, concentration, or amount, including, but not limited to, transcription factor NF-κB, interleukins such as IL-1β, IL-2, IL-6, IL-12, transforming growth factor (TGF-β); the presence, concentrations, or amounts of immune cells, including, but not limited to, macrophages, mast cells, neutrophils, basophils, eosinophils, B lymphocytes, T lymphocytes such as Tregs, or natural killer cells; or vaginal fluid viscosity. It may be beneficial to monitor the change in vaginitis (inflammation of the vagina) from any cause, as well as rates of occurrence for an individual. Any change in the vaginal microbiome associated with the use of a medical device, such as a pessary, a condom, a diaphragm, an intrauterine device, a dental dam, a menstrual cup, a vaginal suppository, a tampon, a speculum, a vaginal tissue expander, intravaginal mesh, or other intravaginal device, may also be monitored.

Embodiments of the feminine hygiene system of the present application may be provided in the form of a gel, a lubricant, a wash, a wipe, a suppository, other suitable forms for vaginal application, or combinations thereof (collectively referred to as “product” herein). An embodiment of the female hygiene system may comprise a solvent such as water, dimethylsulfoxide, ethyl alcohol, 3-methoxy-3-methyl-1-butanol (MMB), polyethylene glycol (PEG-400), glycerol, or propylene carbonate; a viscosity (or rheology) modifier which may increase or decrease viscosity of the lubricant, such as hydroxymethylcellulose or other cellulose derivatives, calcium chloride, guar (Cyamposis tetragonoloba) gum or powder, xanthan (Xanthomonas campestris) gum, carob (Ceratonia siliqua) gum, agar, carrageenan, gum arabic, Caesalpinia spinosa gum, Konjac gum, glucomannan, gellan gum, sclerotium gum, tragacanth gum, dehydroxyxanthan gum, acacia gum, tapioca, Atlox Rheostrux 300A™, glycerol, pectin, gelatin, and chitosan; a potassium salt, for example, at up to 0.5%, 0.15-0.35%, 0.01-1.5%, 0.001-1.5%, 0.05-0.55%, 0.1-0.5%, and 0.1-0.3% by weight (optimal is 0.11% by weight of the potassium portion of a potassium salt); a sodium salt, for example, at up to 1.5%, 0.3-1.0%, 0.15-0.35%, or 0.1-1.5% by weight (optimal is 0.19% by weight of the sodium portion of a sodium salt); and a calcium salt, for example, at up to 0.5%, 0.07-0.12%, 0.01-0.12%, 0.1-1.5%, 0.005-0.12%, 0.01-0.2%, and 0.001-1.5% by weight (optimal is 0.05% by weight of the calcium portion of a calcium salt); and has an osmolality, for example, of from 250-450 mOsm/kg (optimal), from 300 to 400 mOsm/kg, from 300 to 450 mOsm/kg, from 290 to 450 mOsm/kg, from 250 to 500 mOsm/kg, from 340 to 360 mOsm/kg, up to 500 mOsm/kg, from 100 to 500 mOsm/kg, or from 50 to 1000 mOsm/kg. The lubricant may further comprise a magnesium salt, for example, at up to 0.5%, 0.001-1.5%, 0.03-.0.07%, 0.01-0.15%, and 0.3-1.0% by weight (optimal is 0.008% by weight of the magnesium portion of a magnesium salt); and/or a manganese salt, for example, at up to 0.1%, 0.001-0.002%, 0.0001-0.001%, 0.002-0.01% by weight (optimal is 0.002% by weight of the manganese portion of a manganese salt). Ions may be added as chloride or other salts. For example, the potassium salt may be potassium chloride (KCl), potassium bicarbonate (KHCO₃), potassium gluconate (C₆H₁₁KO₇), potassium bromide (KBr), potassium fluoride (KF), or potassium iodide (KI); similar anions could also be used with sodium, calcium, magnesium, or other cations.

In some embodiments, the products of the feminine hygiene system may have from 0.03% to 0.07% of the magnesium salt, from 0.15% to 0.35% of the potassium salt, from 0.07% to 0.12% of the calcium salt, and from 0.30% to 1.0% of the sodium salt. The lubricant may be isotonic with human vaginal fluid. In an embodiment, the lubricant has an osmolality from 100-500 mOsm/kg. The lubricant may be non-toxic to the human vagina, not causing excessive cell death or sloughing, not compromising epithelial integrity or disturbing intercellular junctions.

In one embodiment, the products of the feminine hygiene system comprise from 0.15% to 0.35% of the potassium salt, from 0.01% to 0.12% of the calcium salt, and from 0.30% to 1.0% of the sodium salt. Embodiments of the lubricant may be a gel with a pH from 2.9 to 4.6. The lubricant may include up to 2% by weight of lactic acid.

In one embodiment, the products of the feminine hygiene system comprise a ratio of ions that is bio-matched to that of a healthy vaginal biome. Bio-matching means formulating a product with similar (“matched”) levels of specific characteristics as would be found naturally in a healthy vagina, including, but not limited to, pH; lactic acid concentrations or amounts; concentrations, amounts, or ratios of specific ions and/or salts; and presence, concentrations, numbers, or amounts of specific species or genera of bacteria. As there are ranges of what is considered healthy, bio-matching does not necessarily equate to being “bio-identical” for any specific individual. Maintaining a bio-matched acidic pH, proper lactic acid concentrations, and bio-matched concentrations of osmotically active solutes allows healthy vaginal bacteria, such as Lactobacillus spp., to prosper and prevents overgrowth of pathogenic bacteria. There is extensive epidemiological evidence that vaginal Lactobacilli play an essential role in a person's reproductive health. A Lactobacillus concentration of 10 8 colony-forming units per milliliter of vaginal fluid (cfu/mL) is considered healthy, whereas a concentration of 10⁵ cfu/mL (one thousand times lower) is typical of women with bacterial vaginosis^{[8]}. In one embodiment, the products of the feminine hygiene system are bio-matched to both a ˜2-to-1 ratio of sodium to potassium and to a pH range of 3.6-4.2, 2.9-3.7 or 3.8-4.6 to promote optimal concentration of Lactobacilli. The sodium to potassium ratio may also be, for example, 2:1, 2.5:1, 2.7:1, 3:1, or in the range of 1:1 to 3:1. The pH of the products of the feminine hygiene system may also be, for example, 3.5-4.2, 3.5-4.5, 2.5-5.5, 4.2-4.6, 4.6-5.0, 2.9-4.6, 3.0-5.0, 4.0-4.5, 3.7-4.7. The products of the feminine hygiene system may contain lactic acid at amounts of, for example, up to 2%, 0.5-1.0%, 0.5-1.5%, 0.5-2.5%, 0.2-2%, 0.1-0.2%, 0.05-2.0%, 0.01-2.5%, 0.01-3.0%, and about 1%. The lactic acid may have a racemic index, which is represented as a ratio of the L and D forms as L/D, of 0/100, 10/90, 20/80, 30/70, 40/60, 50/50, 60/40, 62.5/37.5, 70/30, 80/20, and 90/10.

Vaginal Lactobacillus spp., the most abundant genus of bacteria in the healthy vagina, are the endogenous source of vaginal lactic acid, a vital source of antimicrobial activity that provides significant protection against vaginal pathogens, including most pathogens that are responsible for sexually transmitted infections, perinatal infections, and other reproductive tract infections. Given the differences in presence and concentrations of vaginal Lactobacilli among women, there are also significant differences in concentrations of vaginal lactic acid. The concentration of vaginal lactic acid associated with a healthy, Lactobacilli dominated vaginal microbiota is between 0.5%^{[9]} and 1.0%^{[10,11]}. In women with bacterial vaginosis, the concentration of lactic acid is significantly lower at approximately 0.1-0.2%^{[12]}. There is extensive epidemiological evidence that this difference is critical. Further, Lactobacillus spp. play a significant role in the health of the epithelium in the vagina. Lactobacillus spp. inhibit binding of other bacteria to epithelial cells and produce lactic acid that kills or inhibits the growth of many damaging bacteria. Lactobacillus spp. are tolerated by vaginal epithelial cells and inhibit induction of pro-inflammatory cytokines by the body's immune system.

In one embodiment, Lactobacillus spp. presence is assessed.

In another embodiment, Lactobacillus spp. concentration is assessed.

In another embodiment, presence of Lactobacillus spp. or other vaginal bacteria is assessed.

In some embodiments, the product has a pH in the range of 3.6-4.2. In some embodiments, the product has a pH in the range of 2.9 to 3.7.

In some embodiments, the product has a pH in the range of 3.8 to 4.6.

In some embodiments, the product has a pH in the range of 2.5 to 5.5.

In some embodiments, the product has a pH in the range of 4.2-4.6.

In some embodiments, the product has a pH in the range of 4.6-5.0.

In some embodiments, a feminine wash may be used to correct the balance of ions. This includes a product for maintaining vaginal health such that a feminine wash may be used to correct the balance of ions should the ion concentration be too low. This includes a product for maintaining vaginal health such that a feminine wash may be used to correct the balance of ions, should the ion concentration be too high. This also includes a product for maintaining vaginal health such that a feminine wash may be used to correct the ratio of ion concentrations, should the ratio of ions become unbalanced when compared to the ion ratio of a healthy vagina.

In some embodiments, a pre-moistened feminine wipe may be used to correct the balance of ions, should the ion concentration be too low. This includes a product for maintaining vaginal health such that a pre-moistened feminine wipe may be used to correct the balance of ions, should the ion concentration be too high. This also includes a product for maintaining vaginal health such that a pre-moistened feminine wipe may be used to correct the ratio of ion concentrations, should the ratio of ions become unbalanced when compared to the ion ratio of a healthy vagina.

In some embodiments, a vaginal topical gel may be used to correct the balance of ions, should the ion concentration be too low. This includes a product for maintaining vaginal health such that a vaginal or topical gel may be used to correct the balance of ions, should the ion concentration be too high. This also includes a product for maintaining vaginal health such that a vaginal or topical gel may be used to correct the ratio of ion concentrations, should the ratio of ions become unbalanced when compared to the ion ratio of a healthy vagina.

In some embodiments, a vaginal homeopathic suppository containing Lactobacillus spp. may be used to correct the microbiome, should the amount or concentration of Lactobacillus be too low.

In some embodiments, assessments of the vaginal microbiome may include a comparison of inflammatory markers indicative of an unhealthy microbiome when compared to a healthy vaginal microbiome. Inflammatory markers may include, but are not limited to, intercellular adhesion molecule (ICAM-1), interleukins (IL-1β, IL-2, IL-6, IL-12) and marker proteins (TGF-β, TNFα, NF-κB). These inflammatory markers are all present in measureable amounts in vaginal secretions.

In some embodiments, the use of isotonic gels may help reduce inflammation and irritation following sexual arousal by helping boost the vagina's natural immune response. This ultimately serves as protection against sexually transmitted infections by encouraging the vagina's natural response to sexual arousal.

In another embodiment, the use of isotonic gels may improve sexual arousal, over time, in women with a history of bacterial vaginosis. This may in turn increase sexual function in women with low sexual arousal.

In another embodiment, the use of isotonic gels may improve vaginal tissue health in women with vaginal dryness due to use of steroidal medications or other drugs; side effects of cancer treatment such as but not limited to pelvic irradiation or chemotherapy; inflammatory conditions such as lichen sclerosis, lichen planus, atrophic vaginitis, desquamative inflammatory vaginitis, genital graft-vs-host disease, pelvic inflammatory disease, contact or other dermatitis, interstitial cystitis, Sjögren syndrome; low hormone levels due to menopause, breastfeeding or stress, pituitary or ovarian defects, hormone suppression, or ovariectomy; cigarette smoking; excessive exercise; and/or depression, and the like.

In another embodiment, establishing a healthy vaginal microbiome by the use of a vaginal probiotic suppository may result in improved sexual arousal by reducing chronic vaginal inflammation.

In some embodiments, determining the state of the vaginal microbiome related to medical conditions such as but not limited to endometriosis, adenomyosis, polycystic ovary syndrome (PCOS), vestibulitis, vaginitis, vaginismus, lichen sclerosis or lichen planus, vulvovaginal candidiasis, vaginal atrophy, vaginal cysts, sexually transmitted infections, peripartum infections, other reproductive tract infections, urinary tract infections, cancers of the female genitalia or urinary tract, interstitial cystitis, and/or pelvic floor disorder (PFD) may be done.

In some embodiments, restoration of the vaginal microbiome to a healthy level after cancer or oncology treatment may be achieved, in addition to ongoing maintenance of the vaginal microbiome after radiation treatment, brachytherapy, chemotherapy, or other iatrogenic causes of unhealthy vaginal microbiome.

In some embodiments, initiation of a vaginal microbiome after creation of a neovagina through gender confirmation surgery may be achieved.

In some embodiments, restoration of a healthy vaginal microbiome during and after menses may be achieved.

In other embodiments, kits are described, wherein a kit comprises means for determining ion concentration in vaginal fluid. In an embodiment a kit comprises means for determining pH in vaginal fluid. A kit wherein the kit comprises means for determining ion concentrations, amounts, and/or ratios in vaginal fluid. A kit wherein the kit comprises means for determining Lactobacillus spp. or other bacterial species concentrations in vaginal fluid. A kit wherein the kit comprises means for determining lactic acid concentrations in vaginal fluid. A kit wherein the kit comprises means for determining secretion viscosity in vaginal fluid. A kit wherein the kit comprises means for determining integrity of the vaginal epithelium. A kit wherein combinations of the above perform multiple assessments and provide multiple products.

Another embodiment describes a system, which includes a step for determining ion ratios in vaginal secretions and correcting imbalances. Once the ion ratio is determined, an analysis of the condition is determined and then the proper product for treatment is dispensed and used. The system may also include a step for determining bacterial ratios in vaginal secretions. Once the bacterial ratio is determined, an analysis of the condition is determined and then the proper product for treatment is dispensed and used.

The system may also include a step for determining lactic acid concentration in vaginal secretions. Once the concentration is determined, an analysis of the condition is determined and then the proper product for treatment is dispensed and used.

The system may include a step for determining pH value and range in vaginal secretions. Once the value and/or range is determined, an analysis of the condition is determined and then the proper product for treatment is dispensed and used.

The system may be a compilation of the steps listed above, in combination in part or in total, for determining health and conditions of the vagina. Once the above are performed, an analysis of the condition is determined and then the proper product for treatment is dispensed and used.

Another embodiment describes a system which is a method to establish and maintain a healthy microbiome in subjects with recurrent bacterial vaginosis. Up to 2 grams of a feminine wash for external use may be applied daily; up to 5-6 grams of a bio-matched intravaginal gel may be applied every-other-day; a single capsule of a probiotic vaginal suppository may be used every third day. Continued use of the system may correct the vaginal microbiome and support improved vaginal health.

Another embodiment describes a system which is a method to establish and maintain a healthy microbiome in subjects with moderate vaginal conditions, including, but not limited to, vaginal atrophy, long-term BV treatment, cancer survivors, and a variety of dermatitis conditions such as lichen sclerosis, lichen planus, interstitial cystitis, and desquamative inflammatory vaginitis (DIV). A feminine wash for external use may be applied daily; a bio-matched intravaginal gel may be applied every-other-day; a probiotic vaginal suppository may be used every third day; a pain gel may or may not be included. Continued use of the system may correct the vaginal microbiome and support improved vaginal health.

Another embodiment describes a system which is a method to establish and maintain a healthy microbiome in women with severe vaginal conditions, women with vaginal damage post-radiation or post-chemotherapy, long-term vaginal or labial pain, severe flares of DIV, lichen sclerosus, lichen planus, and the like. A feminine wash for external use may be applied daily; a vaginal shield may be used; a pain gel may be used as needed. Continued use of the system may allow the vaginal tissue to heal. This system may be combined with another system to restore the vaginal microbiome.

A method of manufacturing a vaginal lubricant includes adding a viscosity modifier, a plurality of salts and an acid, to a solvent, wherein the plurality of salts comprises up to 0.5% by weight of a potassium salt, up to 1.5% by weight of a sodium salt, and up to 0.5% by weight of a calcium salt, so that the lubricant has a pH from 3.0 to 5.0, and an osmolality from 100-500 mOsm/kg. The method may further include adding up to 0.5% by weight of a magnesium salt. The solvent may be water. In one embodiment, the potassium salt is potassium chloride (KCl), the sodium salt is sodium chloride (NaCl), the calcium salt is calcium chloride (CaCl₂), and the magnesium salt is magnesium chloride (MgCl₂). In addition, racemic lactic acid may be added in an amount of from 0.01% to 2.5% by weight.

The potassium salt may be added in an amount of 0.15% to 0.35%, the sodium salt may be added in an amount from 0.15% to 0.35%, the calcium salt may be added in an amount from 0.01% to 0.12%, and the magnesium salt may be added in an amount from 0.30% to 1.0%. The ingredients may be added to achieve an osmolality from 300-450 mOsm/kg. The substance may be iso-osmolal with human vaginal fluid.

In another embodiment, the plurality of salts includes up to 0.5% by weight of a potassium salt, up to 1.5% by weight of a sodium salt, and up to 0.5% by weight of a calcium salt. The salts may further include up to 0.5% by weight of magnesium salt and/or up to 0.1% by weight of manganese salt. In an embodiment, the substance has from 0.03% to 0.07% of the magnesium salt, from 0.001 to 0.002% of the manganese salt; from 0.15% to 0.35% of the potassium salt, from 0.01% to 0.12% of the calcium salt and from 0.30% to 1.0% of the sodium salt. The substance may further comprise 0.002-0.008% by weight of sorbic acid, or from 0.001 to 0.01% by weight of sorbic acid.

Systems and methods are provided herein for bio-matching formulations of the feminine hygiene system (e.g., gels, washes, wipes, suppositories, hydrogels, vaginal shields, serums, pessaries, etc.) to a particular region (or part) of a living body, such as that of a human or other animal. The bio-matching comprises ratios of ions, not only amounts or concentrations. Formulations and methods of formulating thereof may provide compositions that both supplement secretions of the particular region of the living body and promote the health of the particular region. In one embodiment, a method of bio-matching a topical gel is provided. The method may comprise selecting a vagina of a living human body; identifying a secretion of the selected vagina; identifying a composition of the identified secretion; and formulating the topical gel to match the identified composition of the identified secretion. The matching includes using a preselected type and quantity of lactic acid, and formulating to a preselected pH and ion composition (ratio).

In another embodiment, a topical gel for human use is provided. The gel may comprise a formulation matched to a composition of a particular part of a human body. The formulation may include lactic acid, and the particular part may be a vagina or vulva.

In another embodiment, a topical gel for human use may comprise a formulation including lactic acid having a racemic index in a range of about 50% L/50% D. The formulation may be matched to a composition (or chemistry thereof) of a particular part of a human body.

In another embodiment a topical pain/itch gel for human use may comprise a formulation including bio-matched ion ratios and a pain reliever such as lidocaine (up to 4%).

In another embodiment, a vaginal lubricant is provided. The lubricant may comprise a formulation including lactic acid having a racemic index that is bio-matched to a racemic index of natural lubricants in a generally healthy vagina.

In another embodiment, a kit that contains a method for evaluating an ion concentration, ion ratios, vaginal microbiome constitution, lactic acid concentrations, and pH and then recommending a particular system for treatment and maintenance.

In some embodiments, the topical gel may be disposed on a condom prior to the condom being rolled onto the penis. For example, the topical gel may be disposed on the condom (e.g., the outer and/or inner surface) during a manufacturing and/or packaging step of the condom.

In other embodiments, the topical gel may be provided in a package or tube that is separate from a package containing a condom. For example, the topical gel may be provided in a stand-alone container. The user may open the container and apply the topical gel directly to the vagina, vulva, directly to the penis, and/or to any suitable surface of a condom.

In some embodiments, the method may further comprise applying the topical gel to a suitable medical device. For example, the topical gel may be suitable for lubricating one or more implements used during a pelvic exam, such as an outer surface of a glove disposed on a hand of a gynecologist.

A “Recovery” method is provided for subjects (cisgender or transgender) who have had a significant medical challenge or chronic condition that necessitates a sensitive approach to vulvovaginal health. The Recovery method promotes recovery of vaginal tissues and the vaginal microbiome in a vagina with a compromised condition, and includes application of a topical gel and a topical gel including local analgesic to the vagina of a subject and cleaning of the vulva with a wash for promoting recovery of a compromised vaginal condition of a subject. The topical gel may be applied every day, for example, before bed, but may be applied any time of the day. The topical gel with local anesthetic may be applied as needed up to four times a day. The wash may be used daily or as often as the subject bathes or showers. The length of use of the Recovery method and the frequency of use of the topical gel, topical analgesic gel, and vulvar wash may vary based on the condition and the severity of the condition of the vagina, and may be used continuously until the condition is alleviated. The vaginal moisturizing gel may be used every other day or every 3^{rd} day in less severe conditions, while ramping up usage, or for women who are too sensitive to tolerate daily use. Once the condition is alleviated, the Recovery method may be followed by a “Remediation” method and/or a “Stability” method described below in this specification.

In another embodiment, the Recovery method may be combined with cleaning the vulva of the subject with a wipe. The wipe may be used when gentle cleansing of the vagina or vulva is desired between bathing, for example after exercise, during menstruation, after sex, or for other reasons

In another embodiment, the Recovery method may further comprise, when the subject is in need of vaginal procedures, applying a lubricant to the vagina of the subject and/or a medical device such as a speculum or dilator. It may also comprise using a lubricant during sexual intercourse.

In some embodiments, the Recovery method may be used for compromised vaginal conditions caused by medical conditions comprising vulvodynia, vestibulodynia, localized provoked vulvodynia, a vaginal surgery, a neovaginal construction, a transvaginal hysterectomy, a pelvic radiation and/or a brachytherapy, a treatment for a hormone-sensitive cancer, a medically- or disease-induced low-estrogen state, including a use of oral contraceptives, an oophorectomy, adrenal dysfunction, and pituitary or ovarian failure, lichen sclerosus and/or lichen planus, endometriosis, genital graft-vs-host disease, pelvic inflammatory disease, hypertonic or hypotonic pelvic floor disorders, Sjögren syndrome, dermatitis, or combinations thereof.

A “Remediation” method is provided for women who have a dysbiotic condition of a vagina. The Remediation method may include applying a topical gel and a suppository to the vagina of the subject and cleaning the vulva of the subject with a wash. The topical gel may be applied, for example, every other day before bed, and the suppository may be applied, for example, every third day for at least three months, tapering over time until the dysbiotic condition is resolved and suppository is no longer needed. The wash may be used daily or as often as the subject bathes or showers. The suppository may be applied with a small amount of gel to ease insertion or with a full dose of gel or without gel. The suppository may be applied more frequently, such as daily or every other day, in women with no or very low levels of vaginal Lactobacilli, or less frequently in women with moderately low levels of vaginal Lactobacilli, such as every 4, 5, 6, or 7 days. The gel and suppository may be applied before bed, after bathing or showering, first thing in the morning, or at any time of day. The frequency of application and duration of the Remediation method may be adjusted depending on the condition and needs of the subject using the method.

In some embodiments, the Remediation method may be used for dysbiotic conditions caused by medical conditions comprising bacterial vaginosis, recurrent urinary tract infection, recurrent candidiasis, aerobic vaginitis, desquamative inflammatory vaginitis, endometriosis, adenomyosis, lactation-induced vaginal condition, a treatment for a hormone-sensitive cancer, a medically- or disease-induced low-estrogen state, including a use of oral contraceptives, an oophorectomy, adrenal dysfunction, and pituitary or ovarian failure, stress, male-to-female gender transition after gender confirmation surgery, female-to-male gender transition prior to gender confirmation surgery or any similar low estrogen, high testosterone conditions, polycystic ovary syndrome (PCOS), interstitial cystitis, a use of oral contraceptives, sexual trauma, lichen sclerosus, lichen planus, delivery, or combinations thereof.

In some embodiments, the Remediation method may be used for a subject that has previously recovered from a compromised vaginal condition caused by medical conditions comprising vulvodynia, vestibulodynia, localized provoked vulvodynia, a vaginal surgery, a neovaginal construction, a transvaginal hysterectomy, a pelvic radiation and/or a brachytherapy, a treatment for a hormone-sensitive cancer, a medically- or disease-induced low-estrogen state, including a use of oral contraceptives, an oophorectomy, adrenal dysfunction, and pituitary or ovarian failure, lichen sclerosus and/or lichen planus, endometriosis, genital graft-vs-host disease, pelvic inflammatory disease, hypertonic or hypotonic pelvic floor disorders, Sjögren syndrome, dermatitis, delivery, or combinations thereof.

A “Stability” method is provided for women who have a good biome and want help keeping it, or who have improved their biome from either recovery or remediation and want to maintain it. The Stability method may include applying a gel to the vagina of a subject and cleaning the vulva with a wash. The gel may be applied, for example, every day or every other day if there is some vaginal or vulvar dryness; otherwise the gel may be applied 1-3 times daily after triggers for biome disruption such as menses, sex, exercise, swimming, use of biome-disruptive vaginal products (such as douches, antibiotics), oral or intravenous antibiotics, etc. The wash may be used daily or as often as the subject bathes or showers.

In some embodiments, the Stability method may be combined with cleaning the vulva of the subject with a wipe. The wipe may be used when gentle cleansing of the vagina or vulva is desired between bathing, such as after exercise, during menstruation, or after sex.

In some embodiments, the subject using the Stability method may previously have had a condition in which the vaginal biome is disrupted, and has recovered to have the healthy vaginal biome.

In some embodiments, the subject using the Stability method may have previously recovered from a compromised vaginal condition caused by medical conditions comprising vulvodynia, vestibulodynia, localized provoked vulvodynia, a vaginal surgery, a neovaginal construction, a transvaginal hysterectomy, a pelvic radiation and/or a brachytherapy, a treatment for a hormone-sensitive cancer, a medically- or disease-induced low-estrogen state, including a use of oral contraceptives, an oophorectomy, adrenal dysfunction, and pituitary or ovarian failure, lichen sclerosus and/or lichen planus, endometriosis, genital graft-vs-host disease, pelvic inflammatory disease, hypertonic or hypotonic pelvic floor disorders, Sjögren syndrome, dermatitis, or combinations thereof, and/or the subject may have previously remediated a dysbiotic vaginal condition caused by medical conditions comprising bacterial vaginosis, recurrent urinary tract infection, recurrent candidiasis, aerobic vaginitis, desquamative inflammatory vaginitis, endometriosis, adenomyosis, lactation-induced vaginal condition, a treatment for a hormone-sensitive cancer, a medically- or disease-induced low-estrogen state, including a use of oral contraceptives, an oophorectomy, adrenal dysfunction, and pituitary or ovarian failure, stress, male-to-female gender transition after gender confirmation surgery, female-to-male gender transition prior to gender confirmation surgery or any similar low estrogen, high testosterone conditions, polycystic ovary syndrome (PCOS), interstitial cystitis, an use of oral contraceptives, past history of sexual trauma, lichen sclerosus, lichen planus, or combinations thereof.

A method for improving fertility is provided, which includes applying a topical gel, a suppository, and a lubricant to the vagina of the subject, and cleaning the vulva with a wash. The gel may be applied, for example, every other day before bed; the suppository may be applied, for example, every third day; and the lubricant may be applied upon every sexual encounter. When the lubricant is applied, the gel and the suppository may not be applied on the same day but applied on the following day. The frequency of application of the topical gel and suppository may vary depending on the severity of the vaginal microbiome disruption prior to beginning to improve fertility. For example, the topical gel may be applied every day. The suppository may be applied every day or every other day. The gel and suppository may be applied before bed, after bathing or showering, first thing in the morning, or at any time of day. The wash may be used daily or as often as the subject bathes or showers.

A method for alleviating discomfort of a vagina of a postpartum subject is provided. The method may include applying a topical gel, a topical gel with local analgesic to the vulva and/or vagina, and cleaning the vulva with a wash. The gel may be applied, for example, every day before bed beginning one day to one week after delivery, and the gel with local analgesic may be applied, for example, up to four times/day as needed beginning one day to one week after delivery. Gel may be applied before bed, after bathing or showering, first thing in the morning, or at any time of day. The wash may be used multiple times throughout the day beginning as soon as washing the vulva is recommended by a physician.

In one embodiment the method for alleviating discomfort of a vagina of a postpartum subject may include cleaning the vagina or vulva of the subject with a wipe. The cleaning may be performed as needed.

A method for alleviating discomfort of a vagina of a menopausal, a peri-menopausal, or a post-menopausal subject is provided. The method may include applying a topical gel and a suppository to the vagina of a subject, and cleaning the vulva with a wash. The gel may be applied, for example, every day before bed, and the suppository may be applied, for example, every third day, tapering to once per week after two months of usage. Depending on severity of vulvovaginal dryness, the gel may be applied twice a day, once a day, or every other day. The wash may be used daily or as often as the subject bathes or showers.

In some embodiments, the discomfort of a vagina of a menopausal, a peri-menopausal, or a post-menopausal subject is caused by a condition comprising genitourinary syndrome of menopause (GSM), atrophic vaginitis, vulvovaginal atrophy, and menopausal vulvovaginal symptoms including dryness, thin tissues, cracked or ulcerated tissues, burning sensation, pain, or irritation, and combinations thereof.

Application schedules, such as frequency and duration of application and the time of application recited herein are for exemplary purpose only, and not limited to the schedules recited herein. The schedule may be adjusted according to the condition and the daily routine of the subject, and/or by advice of a physician or personnel trained for coaching the use of the feminine hygiene products.

A kit of vaginal care system containing feminine hygiene products is provided for performing the Recovery method. The kit includes a topical gel, a topical gel with local analgesic, and a wash. The kit may further contain a wipe.

A kit of vaginal care system containing feminine hygiene products is provided for performing the Remediation method. The kit includes a topical gel, a suppository, and a wash.

A kit of vaginal care system containing feminine hygiene products is provided for performing the Stability method. The kit includes a topical gel and a wash, and may further include a wipe.

A kit of vaginal care system containing feminine hygiene products is provided for improving fertility of a subject. The kit includes a topical gel, a suppository, a lubricant and a wash.

A kit of vaginal care system containing feminine hygiene products is provided for alleviating discomfort of a vagina of a postpartum subject. The kit includes a topical gel, a topical gel with local anesthetics, and a wash. The kit may further contain a wipe.

A kit of vaginal care system containing feminine hygiene products is provided for alleviating discomfort of a vagina of a menopausal, a peri-menopausal, or a post-menopausal subject. The kit includes a topical gel, a suppository and a wash.

Any one of the kits of feminine hygiene products described herein may have an instruction manual or may be connected with a coaching service, which helps the subject to choose the method or the feminine hygiene products adapted to the subject's conditions and needs.

### BRIEF DESCRIPTION OF THE DRAWINGS

FIG. 1 is a block diagram schematically depicting a composition of an identified secretion of a selected region of a living body.

FIG. 2 is a block diagram schematically depicting a first formulation bio-matched to the composition of FIG. 1.

FIG. 3 is a block diagram schematically depicting a second formulation bio-matched but not bio-identical to the composition of FIG. 1.

FIG. 4 is a block diagram schematically depicting a third formulation bio-matched but not bio-identical to the composition of FIG. 1.

FIG. 5 is a block diagram of a female body, which schematically depicts a composition of an identified secretion of a vagina.

FIG. 6 is a block diagram schematically depicting a first formulation bio-matched to the composition of FIG. 5.

FIG. 7 is a block diagram schematically depicting a second formulation bio-matched but not bio-identical to the composition of FIG. 5.

FIG. 8 is a block diagram schematically depicting a third formulation bio-matched but not bio-identical to the composition of FIG. 5

FIG. 9 is a block diagram schematically depicting the bio-matched formulation of FIG. 6 being applied to an unhealthy vagina of a female user.

FIG. 10 is a block diagram of the female user of FIG. 9 after application of the bio-matched formulation.

FIG. 11 is a flowchart depicting a method of bio-matching a topical gel to a living body.

FIG. 12 shows concentrations of ions in vaginal fluid in mOsm/kg. For magnesium, concentrations are multiplied by 10, and for manganese, concentrations are multiplied by 100 for ease of comparison. Concentrations may fall within the range indicated by error bars.

FIG. 13 shows ratios of ions in vaginal fluid. Na=sodium. K=potassium. Cl=chloride. Mg=magnesium. Mn=manganese. Magnesium is multiplied by 10, and manganese is multiplied by 100 for ease of comparison. Ratios may fall within the range indicated by error bars.

FIG. 14 shows ranges of pH in vaginal fluid for healthy or disease conditions, and for various embodiments.

FIG. 15 shows ranges of ion concentrations in % w/v in embodiments. Magnesium is shown 10 times higher than used in products, and manganese is shown 100 times higher than used in products for ease of comparison.

FIG. 16 shows ratios of ions in vaginal fluid. Na=sodium. K=potassium. Cl=chloride. Mg=magnesium. Mn=manganese. Magnesium is multiplied by 10, and manganese is multiplied by 100 for ease of comparison. Ratios may fall within the range indicated by error bars.

FIG. 17 shows possible ranges of ion concentrations in % w/v in embodiments. Concentrations may fall within the range indicated by error bars.

FIG. 18 shows possible ranges of ion concentrations in % w/v in embodiments. Concentrations may fall within the range indicated by error bars.

FIG. 19 shows potassium, sodium and calcium concentrations in blood, fluid extracted from brain and cardiac tissue, and in vaginal fluid.

FIG. 20 is a block diagram schematically depicting a kit of vaginal care system containing feminine hygiene products.

### DEFINITIONS

An ion is a charged particle that is dissolved in water or another solvent. Ion are also commonly called electrolytes. Ions may consist of a single atom as in the case of sodium or chloride, or may be polyatomic as in the case of bicarbonate or benzoate. Ions may be positively charged (“cations”), such as sodium, potassium, calcium, or magnesium; or they may be negatively charged (“anions”), such as chloride, bicarbonate, sorbate, or benzoate. When a cation and an anion crystallize (come out of solution to form a solid), together they form a salt, such as sodium chloride, potassium benzoate, potassium sorbate, or sodium bicarbonate, for example.

Osmolality and osmolarity—two closely related terms—are defined as measures of how a dissolved substance (“solute”) affects the movement of water. Water molecules tend to distribute themselves with respect to ions or other osmotically active molecules, with water moving into areas with high concentrations of solutes and away from areas with low concentrations of solutes. Additionally, water molecules interact with charged areas on other kind of large, complex molecules; molecules with multiple charged areas tend to ‘draw’ water molecules in a way that molecules without charged areas do not. The osmolality of a product, therefore, is dependent upon both the concentration/quantity of molecules in a product and the specific types of osmotically-active solutes in the product. Osmolality is defined as osmoles of a substance per kilogram of water, while osmolarity is defined as osmoles of a substance per liter of water. Since a liter of pure water has a mass of one kilogram, for our purposes, osmolarity and osmolality are identical concepts.

Hyperosmolal, hyperosmolar, or hypertonic products are defined as those with osmolality higher than the osmolality of vaginal epithelial cells (approx. 290-450 mOsm/kg). Hypertonic products would have an osmolality higher than approx. 500 mOsm/kg. When these products are applied to living cells, water moves out of the cell into the product. This causes the cells to shrink, and quickly die. This effect is believed to underlie the damaging effects of hyperosmolar or hypertonic lubricants.

Iso-osmolal, iso-osmolar, or isotonic products are defined as those with osmolality similar to that of the contents of living cells, approx. 290-450 mOsm/kg. Consequently, when the product is applied to living cells, there is no net movement of water either in or out of the cells. Product components which ensure products are isotonic to vaginal secretions include sodium salts, potassium salts, calcium salts, magnesium salts, or manganese salts.

Hypo-osmolal, hypo-osmolar, or hypotonic products are defined as those with osmolality lower than vaginal epithelial cells. Hypotonic products would have an osmolality less than 200 mOsm/kg. Consequently, when the product is applied to living cells, water moves from the product into the cells. This causes the cells to swell and (theoretically) burst. In practice, however, it is difficult to formulate products to be hypotonic, and living cells are quite good at protecting themselves against hypotonic stresses.

A healthy vaginal microbiome is defined as one with high levels of beneficial bacteria and low levels, or no levels, of pathogenic bacteria, resulting in a vaginal pH between 3.6 and 4.2 or 3.5 to 4.5. Often, but not always, a healthy vaginal microbiome is dominated by one or more Lactobacillus species, including L. crispatus, L. salivarius, L. rhamnosus, L. acidophilus, L. plantarum, L. gasseri, and L. fermentum, among others. Unhealthy vaginal microbiomes may have high levels of Gardnerella vaginosis, Atopobium vaginae, and Megasphaera spp., and/or many others; a healthy vaginal microbiome contains low or no levels of these organisms.

Bio-matching is the method of formulating a product to have similar components as those found in the part of the body for which the product is intended for use. A product bio-matched to the vagina would be isotonic to healthy vaginal secretions. A product bio-matched to the vagina may include sodium, potassium, calcium, and other ions at ratios matching those found in healthy vaginal secretions. A product bio-matched to the vagina may include sodium, potassium, calcium, and other ions at concentrations or amounts matching those found in healthy vaginal secretions. A product bio-matched to the vagina may contain racemic lactic acid consisting of approximately 50% L and 50% D enantiomers of lactic acid. A product bio-matched to the vagina may include Lactobacillus spp. or other bacteria typically found in a healthy vagina. Because there is a wide range of what is considered healthy in terms of pH, lactic acid concentrations, ion ratios, and microbiome constitution, bio-matching to the within the healthy range is feasible, while attempting to produce bio-identical products for individuals is not.

### DETAILED DESCRIPTION

The vaginal microbiome is unique when compared with microbiomes found elsewhere in the body, and is critical to maintaining health of vaginal tissues. The vaginal microbiome is dominated by several species of Lactobacillus, including L. crispatus, L. salivarius, L. rhamnosus, L. acidophilus, L. plantarum, L. gasseri, and L. fermentum. These species, among others, produce racemic lactic acid as an end-product of metabolism of carbohydrates. The chief source of carbohydrates for these bacteria is glycogen, found in large quantities in vaginal fluid. After enzymatic degradation to dimers and small polymers by vaginal cells, bacteria break the glycogen metabolites into individual glucose monomers, then metabolize the glucose to lactic acid to produce the energy they need for survival. Lactic acid may be produced as one of two “enantiomers” (optical isomers—same molecule arranged as minor images of each other), but some bacterial species only produce either D-lactic acid or L-lactic acid. L. crispatus in particular is a beneficial species known to produce both enantiomers of lactic acid (termed “racemic” lactic acid); however, many women lack the beneficial bacteria needed to produce racemic lactic acid and therefore do not have the full protection from pathogenic bacteria they could have.

Accumulation of lactic acid acidifies the vaginal fluid to a pH of approximately 3.5 to 4.2 or 3.5 to 4.5; this highly acid environment is tolerated by the lactic acid-producing bacteria, but prevents growth of other potentially pathogenic bacteria. It is important to note that there is a wide range of what may be considered “normal” or “healthy”, and there is variability in both microbiome species constitution and vaginal fluid pH based on a range of factors, including age, menstrual/menopausal status, and even racial or ethnic background. In addition, the vagina is subject to variations in these based on presence of menstrual blood, semen, or personal care/hygiene products.

Vaginal tissues are accustomed to an acidic environment. Vaginal fluid and cervical mucus which flows into the vagina (together termed “vaginal secretions”) produce some protection for vaginal epithelial tissue from the acid. In addition, vaginal epithelium is a rapidly reproducing tissue. As cells at the luminal (interior) surface die, they slough off, and newly produced cells replace them, much like skin. However, conditions which increase the rate of cell death and sloughing leave the vaginal epithelium with too little protection, and damage to the epithelium can accumulate over time. One such condition is having an imbalance of ions or other osmotically active solutes that dehydrate the vaginal epithelial cells, causing cell death and sloughing.

Sodium and potassium ions operate in concert as essential tools in a living cell's ability to modulate the flow of water, acidity, and electrical charge in and out of the cell. Calcium ions are a necessary counter to sodium and potassium, maintaining the electrical charge balance required to move sodium and potassium to where they are needed. The absolute amounts and relative ratios of sodium and potassium, as well as calcium sodium/potassium may help cell survival, and hence to tissue integrity, bodily function, and defense against infection.

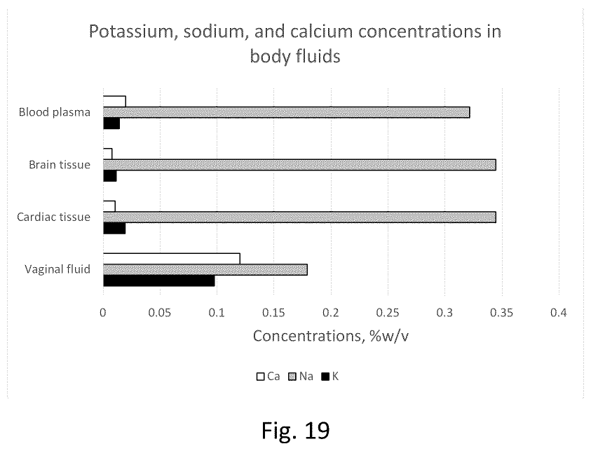

Sodium, potassium, and calcium are provided to every part of the body by the blood. However, the amounts and ratios of these ions are ‘fine-tuned’^{[1,2,3]} in various body tissues, and even small deviations from these local parameters have serious consequences for cellular survival. For example, elevated potassium levels (i.e., lower sodium:potassium ratio) may lead to cardiac arrest, while low sodium:potassium levels may lead to altered mental status due to neuronal dysfunction. New research is showing the importance of dietary sodium:potassium ratios in cardiovascular health^{[13]} and risk of stroke^{[14]}. The local modulations in tissue ion ratios tend to be quite moderate throughout the body, except for the female urogenital tract. The average concentration of potassium in vaginal fluid is five times higher than that of blood, while the average concentration of sodium in vaginal fluid is only half that of blood^{[4,5,6]}. The sodium:potassium ratio of plasma is 35:1, while the sodium:potassium ratio of vaginal fluid is approximately 2.7:1. This distinct composition is necessary for the survival and function of epithelial cells in the challenging vaginal environment, with its broad perturbations of water content and acidity. FIG. 19 summarizes data from references 1-7, comparing the concentrations of potassium, sodium and calcium ions in blood, in fluid extracted from brain and cardiac tissue, and in vaginal fluid. As the figure shows, the ion composition is strikingly different from the other fluids shown, though the relatively subtle differences among the other three fluids are known to have physiological significance. Because of this, bio-matching the sodium, potassium, and calcium content of a vaginal product is not as simple as matching the commonly measured concentrations/ratios of blood, but must involve matching product formulations to the very different and distinct ion concentrations and ratios found in healthy vaginal secretions.

A healthy vaginal microbiome consists of healthy vaginal microbes within healthy vaginal secretions produced by healthy vaginal tissues. A feminine hygiene system which combines vaginal probiotic Lactobacillus species with products that create a healthy environment in which those species may thrive will be more effective than any single product on its own. Furthermore, adapting the feminine hygiene system components and usage protocols to specific vaginal conditions will ensure maximum benefit for women.

A method of formulating bio-matched feminine hygiene products includes using sodium, potassium, calcium, and other ions in appropriate concentrations and ratios; including racemic lactic acid at ˜1.0%; formulating products to be isotonic to vaginal fluid (-290-450 mOsm/kg); and formulating the pH to the range of 3.6 to 4.2. It also includes avoiding potentially damaging ingredients such as glycerin, propylene glycol, and potentially damaging characteristics such as hypertonicity, or a pH above 4.2. For some products, bio-matching also includes providing Lactobacillus spp. found in a healthy vagina, including L. crispatus, L. salivarius, L. rhamnosus, L. acidophilus, L. plantarum, L. gasseri, and L. fermentum.

A system for establishing and maintaining a healthy microbiome includes one or more assessment tools, a set of bio-matched personal hygiene products to correct any characteristics out of the healthy range, and sets of methods of use for combinations of products to support specific needs. The assessment tools may include a test for vaginal fluid pH, a test for vaginal fluid sodium concentrations, a test for vaginal fluid potassium concentrations, a test for vaginal fluid sodium:potassium ratios, a test for total lactic acid levels, a test for D-lactic acid levels, a test for L-lactic acid levels, a test for presence, amounts, and/or concentrations of various bacteria (see list in introduction), a test for vaginal moisture, a test for inflammatory markers, a test for immune cells. The bio-matched personal hygiene products or feminine hygiene products may include lubricants, washes, gels, wipes, hydrogels, vaginal shields, pain/itch gels, balms, tonics, serums, oils, salves, sprays, or probiotic vaginal suppositories. One set of methods of use is to support women with recurrent bacterial vaginosis; a second set of methods of use is to support women with moderate vaginal symptoms; a third set of methods of use is to support women with several vaginal damage.

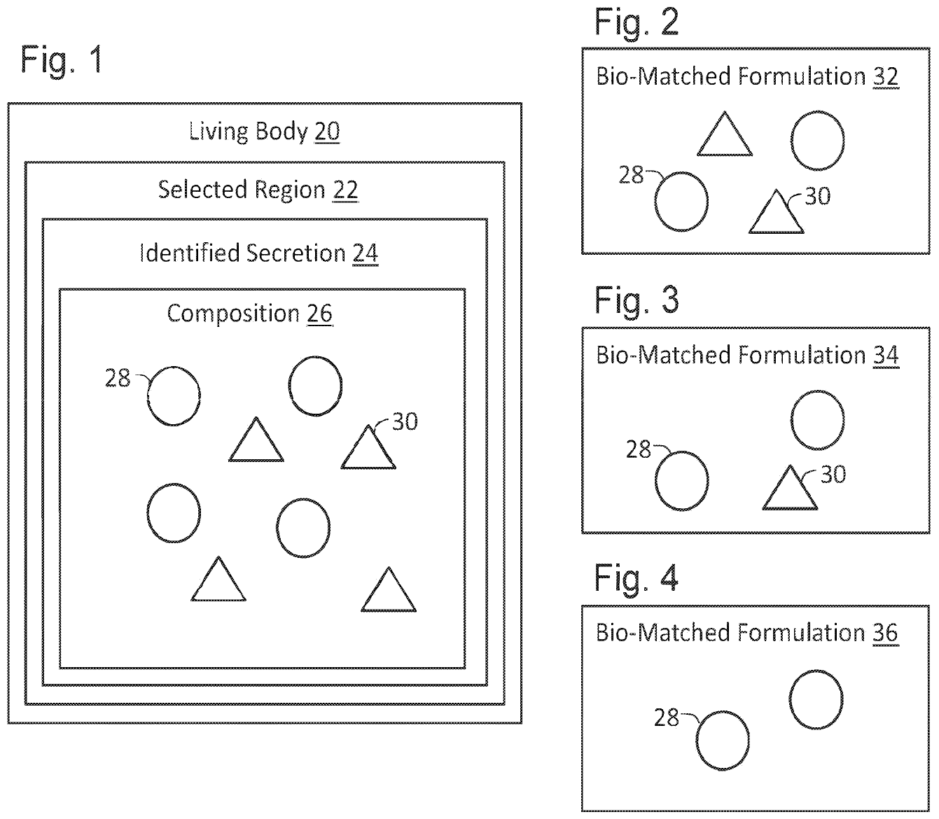

FIG. 1 shows a living body 20, which may be that of a human or other animal In some embodiments, body 20 may be a body of a plant. Body 20 may have one or more regions (or components), such an eye region, an ear region, a vaginal region, a mouth region, and a rectal region among others. A selected region 22 of the one or more regions may produce one or more secretions for one or more purposes (e.g., to produce one or more desired effects). For example, a mouth region may produce saliva to lubricate the mouth region.

The one or more secretions of the selected region may include an identified secretion 24. For example, identified secretion 24 may be a secretion that has been identified as contributing substantially to producing the desired effect (e.g., mouth lubrication, digestion, tartar control, etc.) and/or to promoting the health of the region.

As shown in FIG. 1, identified secretion 24 may include composition 26, which may include one or more chemical compositions, ionic compositions, molecular structures, and/or molecular compositions. For example, the composition may include a first portion of a first type of composition 28, and a second portion of a second type of composition 30. FIG. 1 shows the first and second portions including equal amounts of compositions 28 and 30. In some embodiments, composition 26 may include more than two types of different compositions, and/or may include various ratios of portions thereof.

In some embodiments, body 20 may be a generally healthy body, region 22 may be a generally healthy region, and/or secretion 24 (and/or one or more components of composition 26) may be identified as contributing to the health of region 22 and/or body 20. For example, the composition of region 22 may correspond to a generally healthy composition (e.g., associated with microbiota of a generally healthy, or eubiotic vagina). For example, one or more components of composition 26 or characteristics or properties thereof may be associated with (or present in) generally healthy vagina secretions.

FIG. 2 shows a first formulation 32 that is bio-matched to body 20. For example, formulation 32 may be described as being bio-matched to composition 26. As shown, bio-matched formulation 32 includes a ratio of composition 28 to composition 30 that is equal to the ratio of composition 28 to composition 30 of secretion 24 in FIG. 1.

In some embodiments, formulations bio-matched to secretion 24 may have ratios that are not equal to the ratio of compositions of secretion 24. For example, FIG. 3 shows a formulation 34 having a higher ratio of composition 28 to composition 30 than secretion 24, and FIG. 4 shows a formulation 36 including composition 28 but no composition 30. Formulations 34 and 36 may be described as being bio-matched but not bio-identical to a chemistry (or a composition) of secretion 24 (see FIG. 1).

Formulation 34 and/or formulation 36 may be useful for promoting the health of region 22 (see FIG. 1) and/or the desired effect of secretion 24. For example, composition 28 (or a characteristic of composition 28) may be identified as a significant contributor to the health of region 22 and/or to the desired effect of secretion 24. For example, a higher ratio of composition 28 to composition 30 may be identified as promoting the health of region 22, in which case formulation 34 and/or formulation 36 may be applied to region 22 (or to another body having a corresponding region deficient in composition 28) to increase a supply of composition 28 in that region.

Bio-matching a formulation to body 20 may involve adjusting the formulation to have equal levels of a substance as those found naturally in body 20. For example, ingredients (or compositions) that may be foreign to, produced by, or present in body 20 may be identified as possibly (or actually) detracting from (or harming) the health of region 22 (or body 20 as a whole). In this case, formulations 32, 34, and/or 36 may be bio-matched by avoiding inclusion of one or more of these possibly or actually harmful ingredients (or compositions). Bio-matching comprises assessing the environment of the vagina, including pH, vaginal microbiome, lactic acid amounts or concentrations, ion amounts, concentrations, and ratios, and fluid levels, then comparing those identifiers with that of a type-matched microbiome with regard to race/ethnicity, age, pre- or post-menopause status, etc., and providing a system for treatment or maintenance of vaginal microbiome and the vaginal interior environment.

In some embodiments of bio-matching a formulation to a vagina, harmful ingredients to avoid may include detergents and surface-active agents, glycerol (or glycerin) and other humectant/solvent excipients, one or more preservatives such as chlorhexidine and EDTA, salts or ions in concentrations that make the formulation not isotonic in the vagina, and/or acid in a concentration that does not match that of a healthy vagina.

Typically, portions (or regions) of male and female human bodies secrete various natural substances (or secretions). For example, a portion of a female human body or a male human body (e.g., a gland, organ, or flora associated with the portion or an organ) may secrete or produce one or more particular substances (e.g., lactic acid, saliva, etc.) for one or more particular functions (e.g., lubricating, moisturizing, cell protection, cell repair, protection from pathogens or foreign matter such as dust, etc.).

For example, the female body may include eyes secreting a first substance, a scalp secreting a second substance, ears secreting a third substance, nostrils secreting a fourth substance, a mouth secreting a fifth substance (e.g., saliva), lips of the mouth secreting a sixth substance, armpits secreting a seventh substance, nipples secreting an eighth substance (e.g., pheromones), an epidermis secreting a ninth substance, genitalia secreting a tenth substance (e.g., lactic acid produced by microflora living in the genitalia), a rectum secreting an eleventh substance, and feet secreting a twelfth substance.

The male body may include similar portions secreting (or producing) similar substances. However, due to differences between males and females (e.g., hormonal differences, genetic differences, among others) portions of the male body may differ from portions of the female body, and portions of the male body may secrete substances (e.g., pheromones) that are different than the substances secreted by the female body. For example, the male may include a penis and a scrotum secreting respective thirteenth and fourteenth substances, and a mouth of the male may secrete saliva having a composition that is different than a composition of saliva from the mouth of the female.

The genitalia of the female human body may include labia majora, labia minora, glandula vestibularis major, a urethra, a clitoris, a vestibule, a perineum, a mons pubis, a vagina, a cervix, a uterus, Fallopian tubes, and ovaries. Typically, the labia majora and the labia minora cover the vagina, which leads to the cervix and the uterus; and the urethra runs from the urinary bladder out of the female body, exiting between the labia minora. The vulva comprises the external genitalia, including labia majora, labia minora, clitoris, vestibule, and urethral and vaginal orifices.

A generally healthy vagina may include (or produce, or have present therein) various natural lubricants. Further, a membrane of a vaginal wall of the vagina may produce a second lubricant (e.g., moisture), mucus glands of the cervix may secrete a third lubricant (e.g., different variations of mucus before and during ovulation), and glands, such as glandula vestibularis major located near an opening to the vagina, may secrete a fourth lubricant (e.g., a fluid such as mucus) when the female is sexually aroused.

In particular, mucus from the glands of the cervix and/or moisture from the vaginal wall membrane may provide lubrication within the vagina, and the fluid from the glandula vestibularis major may moisten the labial opening of the vagina, which may make contact with this area more comfortable for the female.

Typically, the female and/or a sexual partner of the female may apply an additional lubricant to components of the genitalia the female, such as the vagina and/or the labia majora and the labia minora, to increase lubrication of the vagina, which may enhance the sexual experience and/or prevent breakage of a condom. For example, the male may roll a condom onto his penis, such that an inside portion of the condom is in contact with the penis. The male may then apply a personal lubricant, such as over the counter K-Y® jelly, to an outside portion of the condom. The female and the male may then engage in sexual intercourse, with the over the counter lubricant providing additional lubrication between the outside portion of the condom and the vagina. However, as previously described, pre-existing lubricants often include components (such as detergents like Nonoxynol-9) that are not bio-balanced to a healthy vaginal environment, and application of such components may make the vaginal environment more prone to disease or damage.

Accordingly, the applicant has discovered that formulating a composition (e.g., topical gel, cream, lubricant, serum, wash, wipe, pain gel, hydrogel, balm, tonic, oil, salve, etc.) to match a composition (e.g., chemical composition, and/or characteristics thereof) of a secretion of a selected region of a human body (e.g., a healthy vagina) may provide or enhance a desired effect (e.g., lubrication) and promote health.

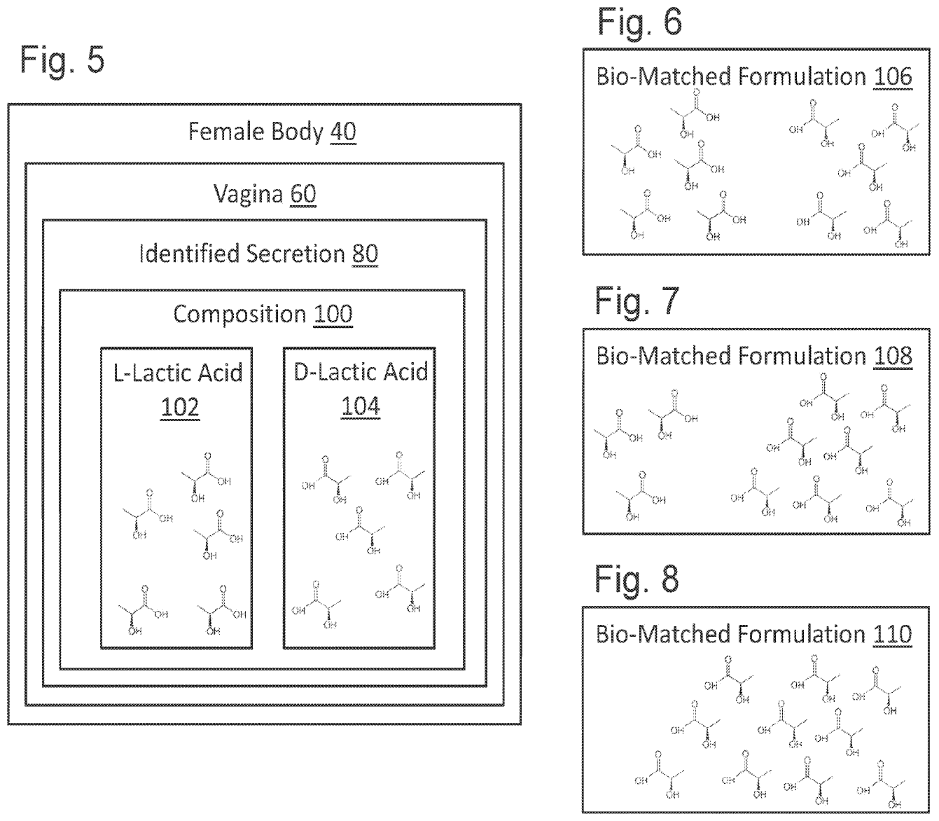

For example, FIG. 5 shows a schematic representation of a female body 40. A vagina 60 of body 40 may secrete one or more substances (or secretions), as previously described. The one or more substances may include an identified secretion 80 having a composition 100. If vagina 60 (and/or body 40) is relatively or generally healthy, then composition 100 may include L-lactic acid. For example, microflora of a generally healthy vagina typically produces lactic acid 102 (i.e., L-enantiomers) and D-lactic acid 104 (D-enantiomers), which are the two optical isomers lactic acid comprising approximately 50% L-lactic acid and 50% D-lactic acid (i.e., lactic acid having a racemic index of about 50% L/50% D). In contrast, it has been found that BV more commonly occurs in vaginas with microflora that produce a large percentage of L-lactic acid and produce only a small percentage (or no percentage) of D-lactic acid.

To provide improved lubrication and vaginal health, applicant has formulated a vaginal lubricant that substantially matches an actual composition and/or characteristic of healthy vaginal secretions and that avoid inclusion of harmful ingredients (e.g., detergents, surface-active agents, glycerol, chlorhexidine, and EDTA). The result is a formulation that is bio-matched to healthy vaginal secretions. Such a bio-matched vaginal lubricant should not include (or avoid inclusion of) any ingredient that might injure healthy vaginal Lactobacilli. Rather, the bio-matched vaginal lubricant may include one or more of the following components and/or characteristics (or properties) that are substantially matched to the components and/or characteristics (or properties) of healthy vaginal secretions:
an aqueous gel component—for example, the bio-matched vaginal lubricant may include a gel that does not include glycerol or other solvents, but only water (or comprises mainly water), as is true for mucus secretions of a healthy vagina;
one or more viscoelastic properties—for example, the bio-matched vaginal lubricant may use a safe and natural polymer to create a gel with viscoelastic properties that match those of vaginal secretions (e.g., mucus) of a healthy vagina, which may include not only matching a viscosity of the vaginal secretions at a given shear rate, but also across a broad range of shear rates—more specifically, mucus of the vagina is a “shear-thinning” lubricant (e.g., a gel that becomes very slippery, and has a low viscosity when the gel is being sheared, as in the act of intercourse), and matching the viscoelastic properties of the vaginal lubricant to the mucus of the vagina may provide for the gel of the bio-matched vaginal lubricant not dripping out of the vagina, but remaining in the vagina and becoming very slippery with a low viscosity only when being sheared;
an isotonic property—for example, the bio-matched vaginal lubricant may be formulated to have a composition of salts, sugars, and other osmotically active molecules that makes the bio-matched vaginal lubricant isotonic in the vagina (e.g., when the bio-matched vaginal lubricant is in the vagina, the bio-matched vaginal lubricant will not cause water to be secreted into the vagina, nor cause water to be absorbed out of the vagina);
an isotonic property that matches as close as possible the ions present in a vaginal secretion (the ions in a vaginal secretion include sodium, potassium, calcium, magnesium, and manganese). These ions must be present at the correct ratios. For example, the sodium:potassium ratio should be 2.7:1.
a pH property—for example, the pH of the bio-matched vaginal lubricant may be formulated to closely match the pH of a healthy, or eubiotic vagina (e.g., pH 3.6-4.2);
a lactic acid component—for example, the bio-matched vaginal lubricant may be acidified with ˜1±0.95% lactic acid, which is a concentration that may match that of a healthy vagina; and
a racemic lactic acid component—for example, the bio-matched vaginal lubricant may include lactic acid that is an essentially equal mix of the D- and L-lactic acid to match the mixture of these enantiomers in a healthy vagina.

With respect to achieving the desired bio-matching property recited above, applicant has learned, by extensive review of research into cell vitality, reproductive tract microbiology and epidemiology of reproductive tract infections, it is particularly important to bio-match osmolality to a range of 250-500 mOsm/kg, pH to a range of 3.5 to 4.5, and racemic lactic acid to a range of 0.05-2.0% by weight. The isotonic property recited above involves an ion composition that is isotonic with vaginal fluid. Isotonicity may be achieved with variations in sodium and potassium salts as well as with other osmotically active compounds.

Osmolality of healthy vaginal secretions has been measured, and using that measurement, the invention includes a gel that bio-matches osmolality to that of a healthy vagina. Based upon that bio-matching, the invented lubricant, including a gel version, may be as close to being isotonic as is possible. Bio-matching, that is, matching the osmolality of the product to the osmolality of vaginal secretions, achieves isotonicity, given the absence of any direct observations of fluid movement into or out of the vagina. In some embodiments, the invented lubricant is isotonic, that is, for example, the gel version is effective to lubricate a human vagina that is neither: (i) hypertonic (causes the vaginal epithelium to secrete water that dilutes the lubricant which can cause cell death of the vaginal epithelium), nor (ii) hypotonic (causes water to be absorbed from the fluid into the vaginal epithelium, causing cells to swell).

Further, the applicant has found that formulating a vaginal lubricant to match the vaginal acidity of a healthy vagina, particularly by including lactic acid in the formulation, and more specifically by including lactic acid that is substantially racemic, kills HIV and many other pathogens.

Recent studies have established harm caused to vaginal epithelial tissue by personal lubricants. Most of the widely used vaginal lubricants in the U.S. and Europe are strongly hypertonic, formulated with high concentrations of glycerol, propylene glycol, polyquaternary compounds or other ingredients that make these lubricants 4 to 30 times the osmolality of healthy vaginal fluid. For example, standard K-Y® Jelly is moderately hypertonic with an osmolality of 2500 mOsm/kg (at the low end of personal lubricants), while K-Y® Warming Jelly has an osmolality of 10,300 mOsm/kg (at the high end of personal lubricants); this is compared with a range of 290-450 mOsm/kg for healthy vaginal fluid. Hypertonic formulations have been shown to cause marked toxicity to human colorectal epithelia in vivo, and significantly increase vaginal transmission of genital herpes infections in the mouse/HSV model. They also cause toxicity to explants of vaginal epithelia, to cultured vaginal epithelial cells, and increase susceptibility to HIV in target cells in cell cultures.

Hypertonic lubricants induce greater epithelial damage than hypo- and iso-tonic lubricants. Given the level of breach in the epithelial barrier by hypertonic lubricants, there is potential of an increase in susceptibility to sexually transmitted infections such as HIV and HSV in individuals who are regular users of hypertonic lubricants. Such effects could be attributed to reduction in barrier integrity of the epithelium as measured by reduced transepithelial electrical resistance, which makes the epithelium “leaky” to allow viral and microbial entry, and alteration of the microbiota in the vaginal environment.

Hypertonic personal lubricants such as KY Jelly®, and the surfactant N9 have been found to be toxic to the beneficial Lactobacilli spp. that can help protect against infections by acidifying the vagina with lactic acid (a broad antiviral and anti-bacterial agent). While toxicity of hypotonic agents is minimal, hypertonic concentrations of glycerol and propylene glycol cause obvious toxicity that increases markedly as the osmolality increases. An optimal range of Lactobacilli spp. to promote vaginal health is in the range of 1×10⁶ to 1×10⁸ cfu/mL cervicovaginal lavage.

Lubricants containing glycerin/glycol, propylene glycol, and polyethylene glycol (PEG-8) as one of the top four ingredients are associated with marked reduction in barrier properties and tissue morphological damage. The presence of glycerin as an ingredient may also contribute to an increase in osmolality which results in the loss of the apical layer in vaginal epithelial tissues exposed to such over the counter products such as RepHresh®. The reduction in barrier function has been shown quantitatively by a reduction in trans-epithelial electrical resistance (TEER).

Historically, the significance of a highly toxic agent, the detergent N9, was not adequately realized until after performing large HIV prevention trials using N9. N9 was, and still is, used as a vaginal contraceptive. Despite its significant toxic effects, it does not cause obvious pain or discomfort in most users.

Similarly, hypertonic lubricants cause little or no obvious pain or discomfort to most users. However, they have been shown to be toxic nonetheless, and to increase susceptibility to sexually transmitted infections. Sexual lubricants have been associated in several studies with increased risk of episodes of bacterial vaginosis, and most sexual lubricants are hyperosmolal with respect to the osmolality of healthy vaginal fluids. Hypertonic vaginal lubricants disrupt barrier functions of the basal and parabasal layers and shedding of the apical layers of the vaginal epithelium, which suggest osmolality-induced disruption of epithelial barrier may be one of the mechanisms by which use of vaginal lubricants is associated with the risk of bacterial vaginosis and may increase susceptibility to sexually transmitted infections.

FIG. 6 shows a first formulation 106 that is bio-matched to a chemistry of body 40 of FIG. 5 (e.g., to a chemistry of secretion 80 of vagina 60). For example, FIG. 6 shows formulation 106 including 50% L/50% D racemic lactic acid. In some embodiments, formulation 106 may include lactic acid having a racemic index of about 10% L/90% D. In some embodiments, formulation 106 may include lactic acid having a racemic index of about 20% L/80% D. In some embodiments, formulation 106 may include lactic acid having a racemic index of about 30% L/70% D. In some embodiments, formulation 106 may include lactic acid having a racemic index of about 40% L/60% D. In some embodiments, formulation 106 may include lactic acid having a racemic index of about 60% L/40% D. In some embodiments, formulation 106 may include lactic acid having a racemic index of about 70% L/30% D. In some embodiments, formulation 106 may include lactic acid having a racemic index of about 80% L/20% D. In some embodiments, formulation 106 may include lactic acid having a racemic index of about 90% L/10% D. In some embodiments, formulation 106 may include D-lactic acid, and no L-lactic acid.

In some embodiments, the racemic lactic acid (or lactic acid having another suitable racemic index) may comprise about 1% of formulation 106. For example, the racemic lactic acid may comprise about 0.5% to about 1.5% of formulation 106. In other embodiments, the racemic lactic acid (or lactic acid having another suitable racemic index) may comprise other suitable percentages of formulation 106. In some embodiments, the racemic lactic acid may be synthetically-derived. In other embodiments, the racemic lactic acid may be naturally-derived. Applying formulation 106 to vagina 60 may lubricate vagina 60, and may promote the health of vagina 60.

FIG. 7 and FIG. 8 show respective formulations 108 and 110, which may be considered as bio-matched to composition 100 of secretion 80 of FIG. 5. For example, FIG. 7 shows formulation 108 including lactic acid having a racemic index of 30% L/70% D, which may be suitable for application to a vagina that is slightly deficient in D-lactic acid. FIG. 8 shows formulation 110 including D-lactic acid, but no L-lactic acid, which may be suitable for application to a vagina that has a greater deficiency of D-lactic acid (or does not produce any D-lactic acid at all).

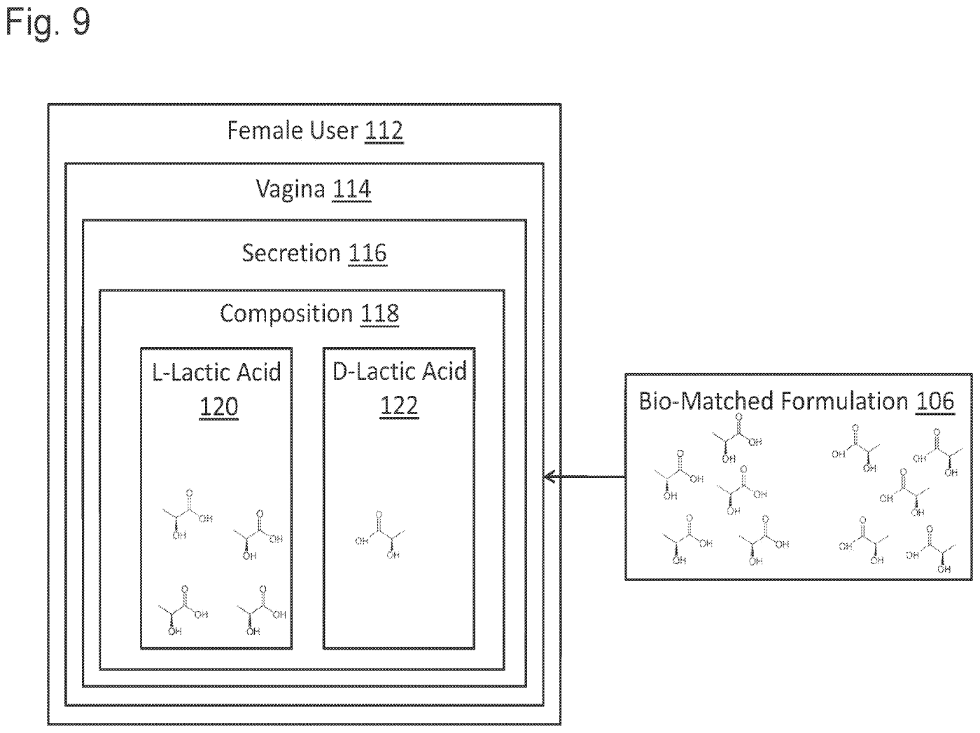

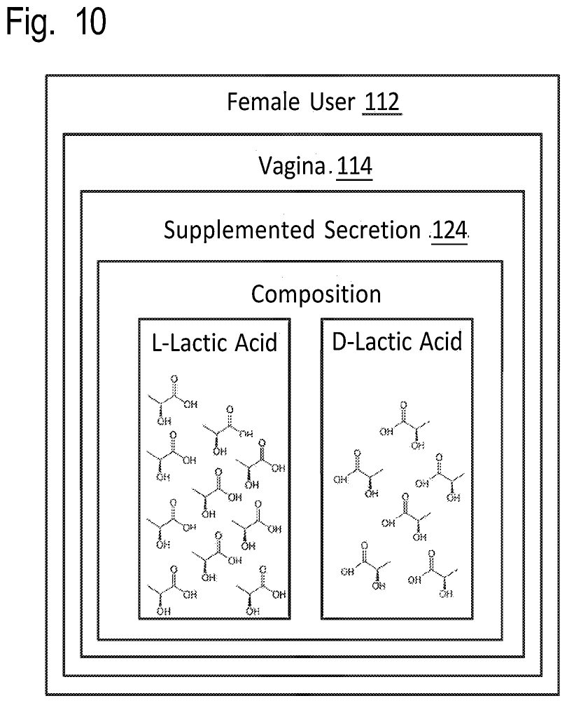

FIG. 9 shows a schematic representation of a female user 112 having a vagina 114 that produces a secretion 116. Secretion 116 may include a composition 118, which may comprise lactic acid including L-lactic acid 120 and D-lactic acid 122. As shown, the lactic acid of secretion 116 has a racemic index of 80% L/20% D, which may be associated with a generally unhealthy condition of vagina 114 (or a condition prone to disease). Application of formulation 106 to vagina 114 may both lubricate vagina 114 and promote the health of vagina 114 (and female user 112). For example, formulation 106 may be added to secretion 116 to produce a supplemented secretion 124 (see FIG. 10).

FIG. 10 shows a schematic representation of female user 112 after application of formulation 106 (see FIG. 9) to vagina 114. As shown, supplemented secretion 124 of vagina 114 includes lactic acid having a racemic index of about 62.5% L/37.5% D, which may more closely match the lactic acid produced by microflora of a healthy vagina.

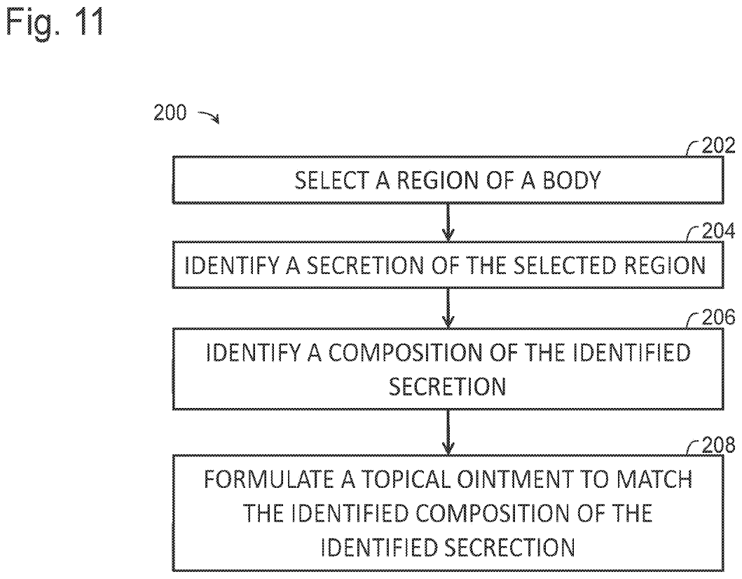

FIG. 11 shows an exemplary method, generally indicated at 200, of bio-matching a topical gel (or cream, lubricant, wash, wipe, serum, hydrogel, pain/itch gel, balms, tonics, oils, salves, or other suitable substance) to a living body. Method 200 may include a step 202 of selecting a region of the body. Step 202 may involve selecting a region including one or more components, such as for example, but not limited to one or more glands, one or more organs, and/or flora (e.g., microflora including bacteria) that secrete various substances. For example, these components may secrete one or more particular substances (e.g., lactic acid, saliva, etc.) for one or more particular functions (e.g., lubricating, moisturizing, cell protection, cell repair, excretion of waste, protection from pathogens or foreign matter such as dust, etc.). The selected body may be a human body, and the selected region may be a vagina. Method 200 may include a step 204 of identifying a secretion of the selected region. Step 204 may involve selecting a secretion produced by (or present in) a generally healthy region, such as a generally healthy vagina. Step 204 may involve identifying a secretion that a relatively healthy body (or part thereof) produces. For example, microflora of a generally healthy vagina typically produces lactic acid comprising approximately 50% L-lactic acid (i.e., L-enantiomers) and 50% D-lactic acid (i.e., D-enantiomers). In contrast, bacterial vaginosis more commonly occurs in vaginas with microflora that produce lactic acid with little or no percentage of D-lactic acid. Accordingly, step 204 may involve identifying lactic acid having L-enantiomers and D-enantiomers. Method 200 may include a step 206 of identifying a composition (e.g., chemical composition, molecular composition, ionic composition, or characteristics or properties thereof) of the identified secretion. Step 206 may involve identifying lactic acid. Step 206 may involve identifying lactic acid having a racemic index in a range of about 50% L/50% D. Method 200 may include a step 208 of formulating the topical gel to (substantially) match the identified composition of the identified secretion. Step 208 may involve selecting lactic acid that is approximately racemic. Step 208 may involve selecting synthetic acid. Step 208 may involve selecting racemic synthetic acid. Step 208 may involve selecting pure racemic synthetic acid having a racemic index of 50% L/50% D. Step 208 may involve formulating the topical gel to include about 1% lactic acid (synthetic and/or racemic). In some embodiments, step 208 may involve bio-matching the topical gel to avoid (or by avoiding) inclusion of one or more ingredients that are toxic (or harmful) to microbiota of the generally healthy vagina (or any vagina). Examples of ingredients that are toxic (or harmful) to the microbiota of a vagina include detergents, surface-active agents, glycerol, many types of preservatives including EDTA, ion concentrations/formulations that make the formulation non-isotonic in the vagina, and acid/base concentrations/formulations that do not match the pH of the generally healthy vagina. Method 200 may further comprise applying the topical gel to the selected region of the human body (or a region of another human body corresponding to the selected region). For example, method 200 may further comprise applying the topical gel to a vagina. For example, applying the topical gel may involve rolling a condom onto a penis, such that an inner surface of the condom contacts the penis; disposing the topical gel onto an outer surface of the condom; and bringing the vagina into contact with the outer surface of the condom. The product may also take the form of a gel or cream packaged in a plastic applicator, a gel or cream packaged with a plastic applicator, a gel or cream packaged to be applied using the fingers, a gel or cream formulated in a dissolving pessary, a gel or cream formulated in a dissolving film.

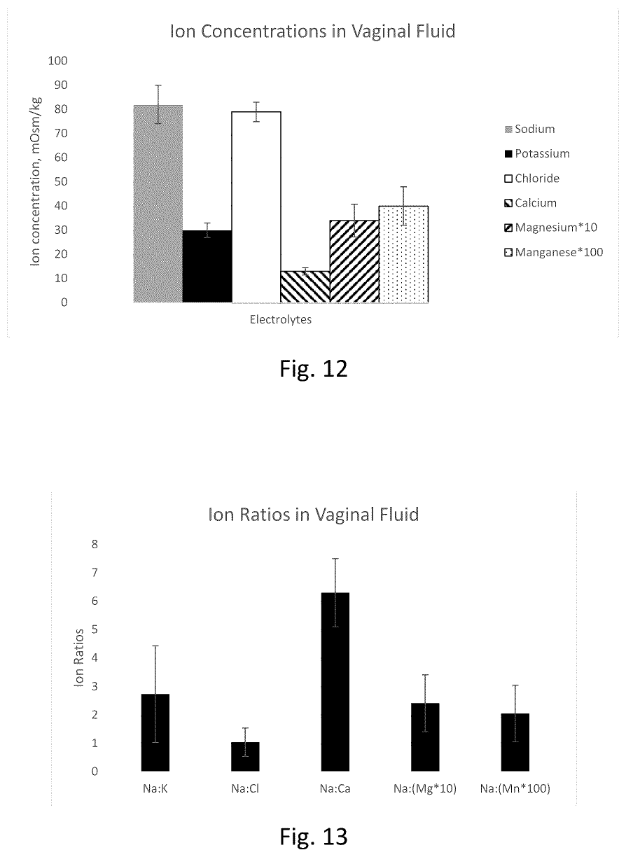

FIG. 12 shows the concentrations of various ions in vaginal fluid disclosed in References 4-6. Sodium, potassium, chloride, and calcium are shown as mOsm/kg. Magnesium is shown as 10 times mOsm/kg and manganese is shown as 100 times mOsm/kg for easier interpretation of the graph because their levels are much lower in vaginal fluid compared with the other ions. Reference 6 measured vaginal fluid ion concentrations in women across the menstrual cycle, showing a wide range but little cycle-dependent variation except for calcium which peaks during ovulation. Reference 4 assessed vaginal fluid composition in women before, during, and after sexual arousal; it showed no distinct pattern of ion composition changes based on arousal.

FIG. 13 shows ratios of sodium to other ions in vaginal fluid, calculated from disclosures in References 4-6. Other ions include potassium, chloride, calcium, magnesium, and manganese. Magnesium is shown as sodium-to-10 times magnesium, and manganese is shown as sodium-to-10 time manganese for ease of graph interpretation because the levels of magnesium and manganese are much lower compared with other ions.

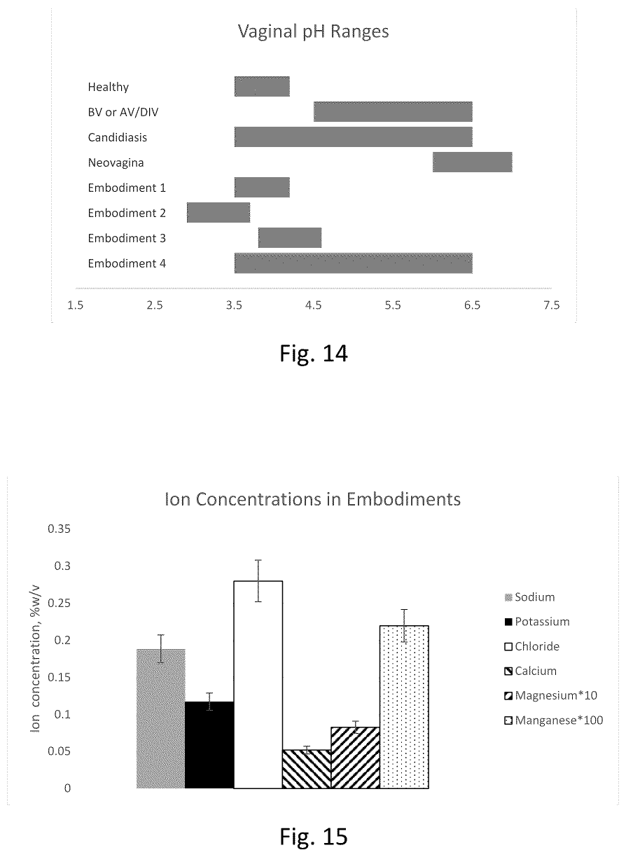

FIG. 14 shows the pH of vaginal fluid in various conditions and in some embodiments. Conditions include healthy, bacterial vaginosis (BV), aerobic vaginitis (AV)/desquamative inflammatory vaginitis (DIV), and vulvovaginal candidiasis (yeast infection). The pH of a neovagina without any intervention is also shown. Vaginal pH is largely reflective of the state of the vaginal microbiome, and in particular, which bacterial species are present in the highest amounts. Native vaginal Lactobacilli acidify vaginal fluid more strongly (lower pH) than pathogenic bacteria or non-native Lactobacilli. Thus, when native Lactobacilli are present—a heathy state—the vaginal pH is approximately 3.5-4.5. During BV and AV/DIV, pathogenic bacteria are present at high amounts, and Lactobacilli are present in low amounts; this elevates vaginal pH to an unhealthy level: at least 4.5, and sometimes as high as 6.5. Candida yeast species may thrive at any vaginal pH, but over time they tend to raise the pH of vaginal fluid. So they may be present at a “healthy” pH of 3.5 up to an elevated pH of up to 6.5. Neovaginas often lack Lactobacilli because they are derived from scrotal, penile, or other tissues, which do not support Lactobacilli well. Thus, neovaginas may have a pH in the range of 6.0 to 7.0. Embodiments may have a pH matching that of healthy vaginal fluid, or a pH lower than vaginal fluid to correct vaginal fluid with a pH that is too high, or embodiments may have a pH higher than vaginal fluid to correct vaginal fluid with a pH that is too low, or an embodiment may have a pH that is adjustable based on the presence of other fluids in the vagina.

FIG. 15 shows the concentrations of various ions in some embodiments. Sodium, potassium, chloride, and calcium are shown as % w/v. Magnesium is shown as 10 times % w/v and manganese is shown as 100 times % w/v for easier interpretation of the graph because their levels are much lower in vaginal fluid compared with the other ions. The ranges shown here are for maintaining a generally healthy vaginal environment; the ion concentrations Bio-Match those observed in healthy vaginas. Sodium, potassium, calcium, chloride, magnesium, and/or manganese are provided to every part of the body by the blood. In the vagina, these ions move from blood to tissues, then out of tissues into the vaginal fluid. However, the amounts and ratios of these ions are ‘fine-tuned’ in various body tissues, and even small deviations from these local parameters have serious consequences for cellular survival. Thus, it is critical to provide ions—especially sodium, potassium, and calcium—in appropriate amounts as indicated in the graph.

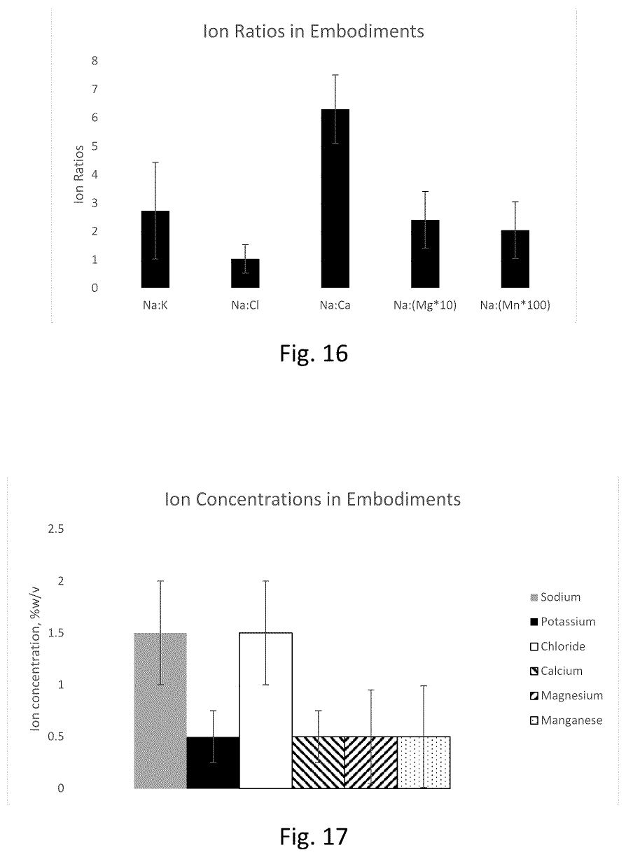

FIG. 16 shows ratios of sodium-to-other ions in some embodiments. Other ions include potassium, chloride, calcium, magnesium, and manganese. Magnesium is shown as sodium-to-10 times magnesium, and manganese is shown as sodium-to-10 time manganese for ease of graph interpretation as the levels of magnesium and manganese are much lower compared with other ions. Ratios may fall within the range indicated by the error bars. Bio-Matching salts in vaginal products is not as simple as matching salt ratios to those found in blood, as vaginal fluid has different salt ratios than blood. Sodium-to-potassium, sodium-to-chloride, sodium-to-calcium, sodium-to-magnesium, and/or sodium-to-manganese ratios are included in embodiments in similar ratios as those ratios observed in healthy vaginal fluid. Sodium transport is used to drive transport of ions other than sodium across cell membranes; this counter-transport of ions is critical for cell survival and health of tissues. Because these counter-transport processes have strict stoichiometry for ions to be transported, which is specific to each transport mechanism, it is not only important to include the correct amounts of ions; it is also important to include the correct ratio of sodium-to-other ions to ensure transport mechanisms continue to work.

FIG. 17 shows possible ranges of ion concentrations in embodiments for sodium, potassium, chloride, calcium, magnesium, and optionally manganese. Concentrations are shown as % w/v and may fall within the ranges indicated by the error bars. If a person's vaginal fluid has low levels of sodium, potassium, chloride, calcium, magnesium, and/or manganese due to a vaginal dysbiosis or other vaginal condition, an embodiment used to treat the dysbiosis or other vaginal condition may include higher levels of one or more of these ions to correct the composition of vaginal fluid. In this way, the embodiment may bring salt concentrations back into the range observed in healthy vaginal fluid.

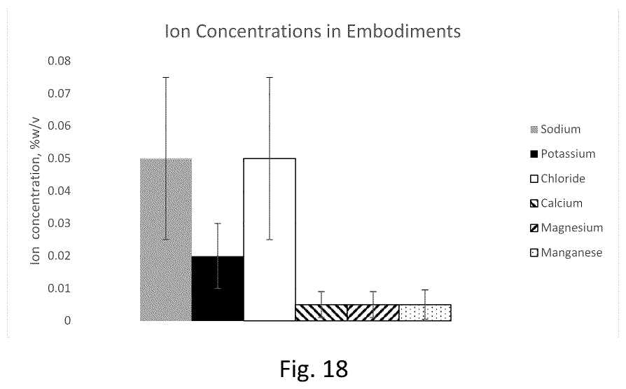

FIG. 18 shows possible ranges of ion concentrations in embodiments for sodium, potassium, chloride, calcium, magnesium, and optionally manganese. Concentrations are shown as % w/v and may fall within the ranges indicated by the error bars. If a person's vaginal fluid has high levels of sodium, potassium, chloride, calcium, magnesium, and/or manganese due to a vaginal dysbiosis or other vaginal condition, an embodiment used to treat the dysbiosis or other vaginal condition may include lower levels of one or more of these ions to correct the composition of vaginal fluid. In this way, the embodiment may bring salt concentrations back into the range observed in healthy vaginal fluid.

FIG. 19 shows sodium, potassium and calcium concentrations in body fluids disclosed in References 1-7. Sodium, potassium, and calcium are provided to every part of the body by the blood. However, the amounts and ratios of these ions are ‘fine-tuned’ in various body tissues, and even small deviations from these local parameters have serious consequences for cellular survival. As can be seen in FIG. 19, the variation of the amounts of calcium, sodium and potassium amounts are moderate among different tissues, except for the female urogenital tract. The average concentration of potassium in vaginal fluid is five times higher than that of blood, while the average concentration of sodium in vaginal fluid is only half that of blood. This distinct composition is necessary for the survival of sperm cells and may well also be necessary for the survival and function of epithelial cells in the challenging vaginal environment, with its broad perturbations of water content and acidity. Therefore, bio-matching the sodium, potassium, and calcium content of a vaginal product cannot be achieved by simply matching the compositions to commonly measured concentrations/ratios of blood, but rather requires matching to different and distinct concentrations/ratios found in healthy vaginal fluid.

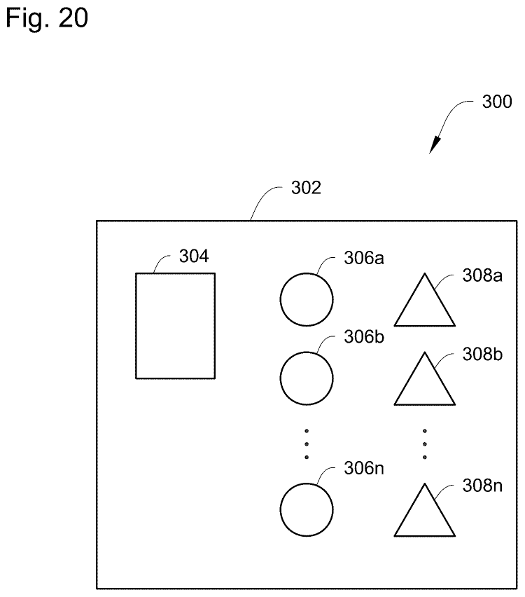

FIG. 20 shows a schematic diagram of a kit of vaginal care system. A kit of vaginal care system 300 may be packaged in a suitable container 302 with a product insert 304 and components 306a-306n. The kit 300 may further contain additional items 308a-308n. The container 302 may be conspicuously labeled or discretely labeled and is configured for easy arrangement and identification of the components. The product insert 304 contains information including, but not limited to, a description of conditions that may be treated by the kit, treatment regimen, such as frequency, time, amounts of compositions to be used, how to apply the compositions, and how to determine whether treatment should be terminated or transitioned into different treatments. The product insert 304 may also contain contact information of the supplier and information of a coaching service. Components 306a-306n are each a feminine hygiene product. Each component is clearly labeled so that each component may be easily distinguished from one another and packaged so that sufficient amount of components is supplied to complete a predetermined treatment regimen for a predetermined period of time. Additional items 308a-308n may include, but not limited to, applicators, disposal containers, bags, envelops, mailing instructions, paraphernalia, and ornamental items.

### EXAMPLE 1

The following shows the preparation of a feminine hygiene product, which may be a moisturizer, a lubricant or a gel, with percentages by weight of the total formulation shown after each component.

TABLE 1

| | Percentage | |
|---|---|---|
| Component | by weight | Function |
| Water | 94.5-97.5% | Carrier |
| Hydroxyethyl Cellulose | 0.1-5.0% | Binder |
| Xanthan Gum | 0.1-2.0% | Binder |
| Ceratonia Siliqua (Carob) | 0.1-2.0% | Binder |
| Gum | | |
| Potassium Sorbate | 0.1-1.0% | Preservative |
| Sodium Benzoate | 0.01-1.0% | Preservative |
| Calcium Chloride | 0.1-1.5% | Astringent |
| Sodium Chloride | 0.1-1.5% | Osmolality Modifier |
| Hyaluronic Acid | 0.1-4.0% | Skin Conditioner |
| Carrageenan | 0.1-1.2% | Binder |
| Lactic Acid | 0.1-4.0% | pH Adjuster |
| Potassium Chloride | 0.01-1.5% | pH Adjuster |

Batches of the product were prepared by combining water, hydroxyethyl cellulose, and xanthan while stirring vigorously. Through multiple temperature cycles and while continuing to stir, all other materials were added to create a gel. It was determined that the product prepared according to the above formulation may be combined to create gels with a pH within the range of 3.0-5.0, and an osmolality of 100-500 mOsm/kg. The osmolality value may be adjusted by varying the amount of salts, and the pH may be adjusted by varying the amount of acid. This may include L-lactic acid, D-lactic acid, or mixtures of L- and D-lactic acid in varying ratios. Viscosity may be adjusted by varying the amounts or types of binders. Binders may be substituted with other gums such as, but not limited to, agar, acacia gum, konjac, chitosan, and guar gum.

According to recently published test results, healthy vaginal secretions have an osmolality of 370+/−40 mOsm/kg, which is higher than the osmolality of most other bodily fluids, which is about 290+/−10 mOsm/kg. The osmolality of this above-identified version of the disclosure is 250-400 mOsm/kg.

The hydroxyethylcellulose is included as a viscosity modifier, or gelling agent. A gel provides lubricity as well as cohesion between molecules in a lubricant, which helps the lubricant or moisturizer to remain in the vagina during intercourse or during treatment of an unhealthy vaginal condition. When large amounts of viscosity modifier are present, formulations become tacky and lose lubricity. Persons of skill in the art will recognize that it is possible to use one or more viscosity modifiers other than hydroxyethylcellulose to create a non-toxic biocompatible gel. That secondary gelling agent may be carrageenan, a nontoxic material made from seaweed. Utilizing carrageenan, for example, may cause the formulation to have the added feature of tending to minimize herpes simplex virus (HSV) and human papillomavirus (HPV) infections. These may be used to increase viscosity: hydroxymethylcellulose or other cellulose derivatives, calcium chloride, guar (Cyamposis tetragonoloba) gum or powder, xanthan (Xanthomonas campestris) gum, carob (Ceratonia siliqua) gum, agar, gum arabic, Caesalpinia spinosa gum, Konjac gum, glucomannan, gellan gum, sclerotium gum, tragacanth gum, dehydroxyxanthan gum, acacia gum, tapioca, Atlox Rheostrux 300A™, glycerol, pectin, gelatin, chitosan. No additive is used to decrease viscosity, since the solvent already has low viscosity. Accordingly, embodiments are not limited to the specific viscosity modifiers provided in the examples.

The primary salt ions used are Na+, K+, Cl−, and Ca++, and that same combination is found in the vagina. While the concentrations of these four ions in some embodiments are not identical to those in a healthy vagina, the combination of these four ions have been adjusted such that this formulation has an osmolality that matches the osmolality of healthy vaginal secretions. Secondary ions used may include Mg++ and/or Mn++; when included, these are also adjusted so that this formulation has an osmolality that matches the osmolality of healthy vaginal secretions. When adjusting concentrations, all salt ions are adjusted to maintain the appropriate ratios of ions to each other.

Bio-matching ion content to healthy vaginal fluid has numerous benefits. In addition to providing a healthy osmolality, bio-matching ion content minimizes disruption of natural conditions to which bacterial flora are adapted. Introducing a substance that alters natural conditions can be harmful to Lactobacillus and other vaginal flora. Accordingly, bio-matched ion content promotes general health. Vaginal fluids are fluids that are present in a normal healthy vagina, and may comprise secretions from the endocervix and the vaginal epithelium.

Measured data suggests that healthy vaginal fluids have significant quantities of potassium, sodium, calcium, manganese, and magnesium. Embodiments of a bio-matched product may include potassium as potassium chloride (KCl), sodium as sodium chloride (NaCl), calcium as calcium chloride (CaCl₂), manganese as manganese chloride (MnCl₂) and magnesium as magnesium chloride (MgCl₂). Embodiments may include up to 0.5% by weight of a potassium salt, up to 1.5% by weight of a sodium salt, up to 0.5% by weight of a calcium salt, up to 0.5% by weight of manganese salt, and up to 0.5% by weight of magnesium salt. In some embodiments, the salts may be included in a range of from 0.03% to 0.07% of the magnesium salt, from 0.001 to 0.002% of the manganese salt, from 0.15% to 0.35% of the potassium salt, from 0.005% to 0.12% of the calcium salt, and from 0.30% to 1.0% of the sodium salt. The vaginal lubricant may be bio-matched to a 1:1 to 3:1 ratio of sodium to potassium.

Some embodiments have concentrations of ingredients that vary from the concentrations provided in the table above. Sodium chloride may be provided in concentrations from 0.3-1.0% or 0.35-0.85%, potassium chloride may be provided in concentrations from 0.1-0.5% or 0.1-0.3%, calcium chloride may be provided in concentrations from 0.01-0.20% or 0.01-0.12%, magnesium chloride may be provided in concentrations from 0.01-0.15% or 0.03-0.07%, manganese chloride may be provided in concentrations from 0.0001-0.001% or 0.002-0.01%, sorbic acid may be provided in concentrations from 0.001-0.01% or 0.002-0.008%, and lactic acid may be provided in concentrations from 0.01-3.0% or 0.05-2.0%.

Additional embodiments have concentrations of ingredients from the above table, where the ratio of sodium to potassium is bio-matched to the ratios naturally present in the vagina. The example includes specification of sodium and potassium ions in a ratio mimicking that in vaginal fluid, a ratio quite unlike that of uterine fluid, semen, or other body fluids. The sodium-to-potassium ratio of vaginal fluid supports viability and function of vaginal epithelial cells in the particular low-pH environment of the vagina and is therefore an enhancement in the formulation of a vaginal lubricant. The example specifies sodium and potassium ions as necessary, in combination and at a fixed ratio, with the concentration of each dependent on the concentration of the other. No other ion or ions may be substituted to provide the same function.

### EXAMPLE 2

The following shows the preparation of a product with a topical analgesic, which may be a moisturizer, a lubricant or a gel, with percentages by weight of the total formulation shown after each component.

TABLE 2

| | Percentage | |
|---|---|---|
| Component | by weight | Function |
| Water | 65.0-80.0% | Carrier |
| Aloe Barbadensis Leaf Juice | 0.25-0.75% | Lubricant |
| Hydroxyethyl Cellulose | 0.3-0.8% | Viscosity Modifier |
| Xanthan Gum | 0.1-0.6% | Binder |
| Lidocaine | 3.0-5.0% | Analgesic |
| Propanediol | 14.0-22.0% | Preservative, Humectant |
| Dimethyl Isosorbide | 1.0-3.0% | Solvent, Carrier |
| Botanical extracts | 1.0-15.0% | Antibacterial, anti- |
| | | inflammatory, antioxidant, |
| | | antiseptic, antifungal, |
| | | natural preservative, |
| | | astringent |
| Sodium Benzoate | 0.05-0.55% | Preservative |
| Potassium Sorbate | 0.05-0.55% | Preservative |
| Lactic Acid | 0.5-1.5% | pH Adjuster |

Batches of the product were produced by vigorously mixing water and Aloe barbadensis, while adding hydroxyethyl cellulose and xanthan gum. Through multiple temperature cycles, other ingredients, including the active ingredients, were added and mixed thoroughly. The composition according to the above formulation may be combined to create lubricants, moisturizer, or a gel with a topical analgesic with a pH within the range of 3.0-5.0, and an osmolality of 100-500 mOsm/kg. The pH may be adjusted by varying the amount of acid. This may include L-lactic acid, D-lactic acid, or mixtures of L- and D-lactic acids in varying ratios. Viscosity may be adjusted by varying the amount or types of binders, including substituting the listed binders with other gums such as agar, acacia gum, konjac, chitosan, and guar gum.

### EXAMPLE 3

The following shows the preparation of a feminine wash, following a procedure as described in connection with Examples 1 and 2, and with percentages by weight of the total formulation shown after each component.

TABLE 3

| | Percentage | |
|---|---|---|
| Component | by Weight | Function |
| Water | 60.0-70.0% | Carrier |
| Aloe Barbadensis | 0.1-5.0% | Moisturizer |
| Leaf Juice | | |
| Cocamidopropyl | 3.0-20.0% | Conditioning Agent |
| hydroxysultaine | | |
| Plantapon | 10.0-35.0% | Surfactant, Preservative |
| Ninol CAA | 0.01-1.0% | Surfactant |
| Natural Lavender Flavor | 0.1-2.0% | Flavor |
| Lactic Acid | 0.25-4.0% | pH adjuster |
| Lamesoft PO 65 | 0.5-4.0% | Viscosity Modifier |
| Botanical extracts | 0.01-3.0% | Antimicrobial, antioxidant, |
| | | skin conditioner, antiseptic, |
| | | antifungal |
| Potassium sorbate | 0.1-1.0% | Preservative |
| Sodium Benzoate | 0.1-1.0% | Preservative |

Batches of feminine wash are prepared by combining water and preservatives with the aloe, while mixing vigorously and heating. A premix tank is used for premixed materials and botanicals. Once each is thoroughly blended, the premixed tank is added to the main tank and blended to homogeneity. Any remaining materials are added and blended. For use on subject with sensitive or irritated vaginal condition, the feminine wash may be prepared without Aloe leaf juice or other ingredients. The feminine wash may be directly used on a subject or may be impregnated into a disposable material and be used as a wipe.

Healthy vulvar and vaginal fluids have an acidic pH, in the range of 3.5-4.2. This feminine wash for external use contains lactic acid in an amount necessary to adjust pH of the product to bio-match this range. The feminine wash has a pH of, for example, 4.0-4.5 or 3.7-4.7. The lactic acid is used to adjust pH, at a concentration of, for example, 0.24-4.0% or 0.4-4.0%.

Harsh ingredients such as sodium lauryl sulfate and hypertonic formulations of pre-existing products can irritate vulvar and vaginal tissues. This feminine wash uses a base of aloe gel, which has soothing properties, and uses cocamidylpropyl hydroxysultaine as a gentle cleanser in place of harsh chemicals.

Bio-matching ion content to healthy vaginal fluid has numerous benefits. In addition to providing a healthy osmolality, bio-matching ion content minimizes disruption of natural conditions to which beneficial bacterial flora are adapted. Introducing a substance that alters natural conditions can be harmful to lactobacillus and other vaginal flora. Accordingly, bio-matched ion content promotes general health. Vaginal fluids are fluids that are present in a normal healthy vagina, and may comprise secretions from the endocervix and the vaginal epithelium.

Additional embodiments may have concentrations of ingredients from the above tables 1-3, where the ratio of sodium to potassium is bio-matched to the ratios naturally present in the vagina. Additional embodiments may include sodium and potassium ions in a ratio mimicking that of vaginal fluid, a ratio quite unlike that of uterine fluid, semen, or other body fluids. The sodium-to-potassium ratio of vaginal fluid supports viability and function of vaginal epithelial cells in the particular low-pH environment of the vagina and is therefore an enhancement in the formulation of a vaginal lubricant. When concentrations of the sodium and potassium ions are specified, in combination and/or at a fixed ratio, the concentration of each would be dependent on the concentration of the other. No other ion or ions may be substituted to provide the same function.

### EXAMPLE 4

The following shows the preparation of a vaginal lubricant, with percentages by weight of the total formulation shown after each component.

TABLE 4a

| | Percentage | |
|---|---|---|
| Component | by weight | Function |
| Water | 94.0-98.0% | Carrier |
| Hydroxyethyl Cellulose | 0.1-3.0% | Viscosity Modifier |
| Xanthan Gum | 0.1-1.5% | Binder |
| Carrageenan | 0.1-1.5% | Binder |
| Sodium Chloride | 0.1-1.5% | Osmolality Modifier |
| Lactic Acid | 0.1-4.0% | pH Adjuster |
| Potassium Sorbate | 0.1-1.0% | Preservative |
| Sodium Benzoate | 0.01-1.0% | Preservative |
| Ceratonia Siliqua (Carob) | 0.1-1.0% | Binder |
| Gum | | |
| Potassium Chloride | 0.01-1.5% | pH Modifier |
| Calcium Chloride | 0.001-1.5% | Viscosity Modifier |

Batches of vaginal lubricant were prepared by combining water, hydroxyethyl cellulose, and xanthan while stirring vigorously. Through multiple temperature cycles and while continuing to stir, all other materials are added to create a gel. It was determined that lubricants prepared according to Table 4a may be combined to create lubricants with a pH within the range of 3.0-5.0, and an osmolality of 100 to 500 mOsm/kg. The osmolality value may be adjusted by varying the amounts of salts, and the pH may be adjusted by varying the amount of acid. This may include L-lactic acid, D-lactic acid, or mixtures of L- and D-lactic acid in varying ratios. Viscosity may be adjusted by varying the amount or types of binders. Binders may be substituted with other gums such as agar, acacia gum, konjac, chitosan, and guar gum.

The following table provides an example of a set of ingredients that are present in an embodiment of another vaginal lubricant. Embodiments of the present disclosure are not limited to the specific values and ingredients in the table.

TABLE 4b

| | Percentage | |
|---|---|---|
| Component | by weight | Function |
| Water | 90-99% | Solvent |
| Hydroxyethylcellulose | 0.5-5.0% | Viscosity modifier |
| Sodium Chloride | 0.1-1.5% | Biomatched Salt |
| Potassium Chloride | 0.001-1.5% | Biomatched Salt |
| Calcium Chloride | 0.001-1.5% | Biomatched Salt |
| Magnesium Chloride | 0.001-1.5% | Biomatched Salt |
| Lactic Acid | 0-0.1% | Weak acidic buffer |
| Potassium Sorbate | 0.001-0.1% | Preservative & weak buffer |
| Sodium Benzoate | 0.001-0.1% | Preservative |
| Sorbic Acid | 0.001-0.1% | Preservative & weak buffer |

The primary constituent of the lubricant described by Example 4 is water which acts as a solvent. The water may be deionized or water or water that has otherwise been purified to remove materials that could react with other ingredients or biological fluids. Hydroxyethylcellulose is present as a viscosity modifier that creates a gel and provides lubricity. The listed functions of the ingredients are merely illustrative, and other purposes are possible. Persons of skill in the art will recognize that it is possible to provide additional ingredients, and to substitute known ingredients for the specific ingredients listed in Example 4 in other embodiments. The lubricant may have a pH from 3.0 to 5.0 and a buffering capacity such that adding 5 millimoles of NaOH to 1 gram of the substance increases the pH by at least 1. The osmolality of the lubricant may be biomatched to the osmolality of a vagina, salt content and pH that may be biomatched to vaginal secretions, and a buffer capacity may be formulated to be significantly lower than the buffer capacity of seminal fluids. The formulae of Example 4 do not include any materials that are toxic or otherwise harmful to the vagina at the listed concentrations. In fact, most of the ingredients are naturally present in the human vagina at the listed concentrations.

### EXAMPLE 5

The following shows the preparation of a vaginal probiotic suppository. Percentages by weight of the total formulation are shown after each component. Ingredients are blended to create a dry powder, which is then packaged into a tapioca starch or gelatin capsule. The probiotic may or may not be accompanied by an applicator.

TABLE 5

| | Percentage | |
|---|---|---|
| Component | by weight | Function |
| Ascorbic Acid | 15.0-40.0% | pH Adjuster |
| Blend of homeopathic drugs | 0.1-5.0% | Homeopathic Activity |
| Probiotic Blend | 25.0-50.0% | Live Probiotic Cultures |
| Maltodextrin | 12.0-18.0% | Filler |

The amount of probiotic blend containing Lactobacillus strains may be, for example, 35.0-45.0% or 25.0-50.0%. The amount of the homeopathic blend may be, for example, 0.5-2.0% or 0.1-5.0%. The amount of maltodextrin may be, for example, 10.0-30.0% or 12.0%-18.0%.

Healthy vaginal microbiomes predominantly contain one or more of several Lactobacillus species. Providing these species as a vaginal suppository will inoculate vaginal fluid, establishing growth of beneficial species, and inhibiting growth of pathogenic species. Providing Lactobacilli will also aid in lowering vaginal pH to a healthy range of 3.5-4.2.

In this vaginal probiotic suppository, the following species are included in a homeopathic suppository. The bacterial species include: Lactobacillus acidophilus LA02 (DSM 21717), L. crispatus LCR01 (DSM 24619), L. gasseri LGS06 (DSM 32405), L. fermentum LF08 (DSM 18297), L. plantarum LP01 (LMG P-21021), L. rhamnosus LR06 (DSM 21981), and L. salivarius CRL1328 (DSM 24441). These are considered inactive ingredients for the purposes of this homeopathic formulation. Active homeopathic ingredients include Kreosotum 6C HPUS, Nitricum Acidum 6C HPUS, Thuja Occidentalis 6C HPUS (for vaginal itching and irritation); Pulsatilla Pretensis 6C HPUS (for vaginal odor); and Sepia Officinalis 6C HPUS (for vaginal discharge).

The following Examples 6-26 describe examples in which subjects in need of vaginal treatment were treated with feminine hygiene products disclosed in Tables 1-5. Examples 7-12 are embodiments of the Recovery method, which may also include Remediation and Stability methods. Examples 6, 13-18 and 22 are embodiments of the Remediation method, which may also include Recovery and Stability methods. Example 19 is an embodiment of the method for improving fertility, which may also include Stability, Recovery, and Remediation methods. Example 20 is an embodiment of the method for alleviating discomfort of a vagina of a postparatum or periparatum subject, which may also include Stability, Recovery, and Remediation methods. Examples 24-26 are embodiments of the method for alleviating discomfort of a vagina of a menopausal, a peri-menopausal, or a post-menopausal subject, which may also include Stability, Recovery, and Remediation methods. Application of the methods is not exclusive and multiple methods may be performed successively, depending on the changes in the vaginal condition of the subject.

### EXAMPLE 6

The following describes a trial to test the efficacy of using a vaginal suppository to treat recurrent BV. Women with two or more BV episodes within the past 2 years were surveyed for 11 weeks. Participants were treated with Metronidazole, an antibiotic commonly used to treat vaginal infections, and simultaneously started on a daily external vaginal wash every other day, consisting of an iso-osmolar intravaginal lactic acid-containing gel (pH <4.4), and a vaginal homeopathic suppository in a probiotic base every 3 days. Measurements of vaginal pH, occurrence of BV and occurrence of yeast were taken biweekly. Participants were requested to journal their associated symptoms daily and patient satisfaction was determined via telephone interview, 10-12 months post trial.

Results showed that during the trial no subject had a recurrence of BV. Mean vaginal pH decreased from 4.54 to 4.08, from baseline to study end. Patient journals showed decreased frequencies of pain and irritation (˜2-fold) as well as itching (˜4-fold). 100% of subjects participating in the telephone interview were satisfied with their symptom resolution 10-12 months post trial.

This shows that consistent use of feminine hygiene products that are iso-osmotic and that also support optimal vaginal pH with bio-matched lactic acid, may prevent BV recurrence and also alleviate associated symptoms. This work has important implications for prevention of recurrent BV and is evidence for the importance of maintaining the vaginal microbiome.

### EXAMPLE 7

The following describes a study of vaginal microbiome pre- and post-pelvic radiation therapy to assess the association between the vaginal microbiome and vaginal/vulvar health outcomes in the post-radiation period. Women aged 18 to 75 years with gynecologic, genitourinary, or gastrointestinal cancer requiring radiation or brachytherapy gave written informed consent and were enrolled in the study. Vaginal swabs to test the microbiome were taken pre-treatment, and 12, 24, or 52 weeks after treatment. Vaginal health was assessed by several self-report questionnaires and by physical exam for vaginal length, pH, and the vaginal health index. Use of a vaginal care system by some self-selected women was assessed observationally. This system comprises an external wash for intimate areas, intravaginal moisturizing gel used every other night before bed, and a vaginal homeopathic suppository with probiotics applied every 3rd night before bed. All women also used an intravaginal gel on vaginal dilators according to UCLA standard of care beginning one month after radiation.

Fifteen women were enrolled in the study. Baseline vaginal microbiomes were BV-like or otherwise unhealthy in 13 of the 14 women with valid biome reads. One woman had a healthy vaginal microbiome at the beginning but deteriorated after radiation treatment. One had no valid vaginal microbiome readings to date due to sample failure during assay. Vaginal pH was elevated to higher than 4.5 in all women at all times. Radiation had a destabilizing effect on the vaginal microbiome: before radiation, biomes had 23.5±3.8 (mean±SEM) species, while after radiation, biomes had 30±2.9 species (P=0.01). The Shannon alpha diversity index increased from 4.8±0.8 before radiation to 5.9±1.9 (P=0.15) after radiation. Change in microbial composition was observed after radiation from before radiation, with Anaerococcus more prevalent after radiation than before. Fusobacterium and Campylobacter, genera not often observed in healthy vaginas, were more prevalent before radiation than after radiation.

Conclusions/Implications: This study showed that cancer patients have disrupted vaginal microbiomes prior to beginning radiation treatment and radiation caused further deterioration of the vaginal microbiome.

### EXAMPLE 8

The following describes a study of the effect of using a vaginal care system in reduction of pain in women having localized provoked vulvodynia. Premenopausal women between ages 18 and 52 diagnosed with localized provoked vulvodynia by cotton swab test who have experienced symptoms for at least 3 months without another explanation for pain were enrolled in a clinical study. After obtaining written informed consent, women were randomized to either an intervention arm or a control arm. All women were given routine care, including appointments at baseline, 2 wks, 6 wks, and 3 months. At all visits, all women completed a questionnaire to self-report symptoms, and had a cotton swab test done. At all visits except 2 wks, all women had vaginal swabs collected for microbiome and vaginal pH analysis. Women in the intervention group used a vaginal care system consisting of three products: an external wash for intimate areas every day or as often as she bathes, an intravaginal moisturizing gel every day before bed, and a homeopathic vaginal probiotic suppository every 3rd night before bed. In addition, the lidocaine cream recommended for routine care (control arm) was substituted with a 4% lidocaine gel made from a similar base as the vaginal moisturizing gel for participants in the intervention arm. The gels and wash were matched to healthy vaginal secretions for pH, osmolality, and lactic acid.

### EXAMPLE 9

The following describes a study to observe the effects of using a vaginal care system to establish neovaginal microbiome after gender-confirmation surgery. An open-label longitudinal randomized controlled trial enrolled 40 transgender women in the intervention arm, 10 transgender women in the control arm, and 10 healthy cis women as an additional control group. Transgender women were enrolled prior to gender-confirmation surgery; cis women were enrolled upon recruitment; all gave written informed consent. All had vaginal microbiome and pH testing and completed the vulvovaginal symptoms questionnaire at baseline (day of surgery—glans penis was sampled prior to surgery), 6 wks, and 6 mo. Questionnaire included inquiry on odor and satisfaction. Transgender women in the control group used KY jelly and any soap. Cis women used a vaginal care system: external wash daily, intravaginal gel every other day, and vaginal probiotic suppository every 3 days for 6 months. The aloe-based gel and wash matched healthy cis vaginal fluid for pH, osmolality, and lactic acid. The intervention arm used a modified system: external wash daily and dilators with intravaginal gel up to 4 times per day from day 6 to week 6 after surgery. After a follow-up examination at 6 weeks, they continued daily wash, 2×/day gel with dilation, intravaginal application of gel every other day before bed, and a homeopathic vaginal probiotic suppository containing Lactobacillus crispatus and other strains every 3rd day before bed until 6 months after surgery. The 3-product protocol was extended to 9 months after surgery if vaginal microbiome was not healthy by 6 months. As an optional add-on, some participants also used a wipe impregnated with the same external wash solution for cleaning between showers, either between 6 days and 6 weeks, or from 6 weeks to 6 months. The 3-product protocol with optional wipe was extended to 9 months after surgery if vaginal microbiome was not healthy by 6 months.

Baseline penile microbiomes had higher alpha-diversity and different bacterial taxa than literature reports for healthy cis vaginas; common taxa on penile skin are Corynebacterium, Finegoldia, Anaerococcus, Peptoniphilus, Staphylococcus, and Prevotella, and mean number of species present was 19 (range 3-63). Penile skin pH was 7 at baseline and neovaginal pH remained at 7 at 6 wks in both transgender groups. Three transgender subjects in the intervention group have completed 6 months; in these, neovaginal pH was between 4.0 and 5.8 (4.7+/-0.9; P=0.05 vs. baseline penile pH). In two of the three subjects, microbiome analysis revealed presence of species originating from the probiotic suppository which had not previously been detected in these individuals, including L. salivarius, L. rhamnosus, L. gasseri, L. crispatus, L. acidophilus, L. fermentum, and L. plantarum, indicating that all species included in the probiotic suppository could be incorporated into the neovaginal microbiome, though not all species in all individuals, supporting the use of blends of probiotic strains rather than single strain probiotics.

Conclusions/Implications: Results show that consistent use of a 3-part vaginal care system may colonize the neovagina with probiotic species and establish a neovaginal microbiome that supports vaginal pH close to healthy cis vaginas. Some individuals needed more than 6 months to achieve this effect.

### EXAMPLE 10

The following describes a study to observe the effects of medical oncology treatment and a vaginal care system on the vaginal microbiome. Women age 18-89 diagnosed with a reproductive cancer and prescribed anti-estrogen therapy were enrolled in a clinical study. After obtaining written informed consent, women were randomized to either control arm or intervention arm and seen before starting the study to obtain baseline data, at 3 months, and 6 months. At all visits, all women completed a questionnaire to self-report on vulvovaginal symptoms and were swabbed for vaginal microbiome and pH analysis. Women used a vaginal care system consisting of two products: an external wash for intimate areas every day or as often as she bathes and an intravaginal moisturizing gel every day before bed. In addition, they were given 4% lidocaine in a gel similar to the vaginal moisturizing gel to use as needed up to 4 times daily. The gels and wash were matched to healthy vaginal secretions for pH, osmolality, and lactic acid. Changes in vulvovaginal symptoms, vaginal microbiome, and vaginal pH over time were examined and recorded.

### EXAMPLE 11

The following describes a study to observe the effects of using a vaginal care system on the vaginal microbiome and vulvovaginal symptoms in women with lichen sclerosus. Women between ages 18 and 89 diagnosed with lichen sclerosus were enrolled in a clinical study. After obtaining written informed consent, women were randomized to a control arm or an intervention arm, and were seen before starting the study to obtain baseline, at 3 months, and 6 months. Women in the control group received routine care. Women in the intervention group received routine care and also used a vaginal care system consisting of three products: an external wash for intimate areas every day or as often as she bathes, an intravaginal moisturizing gel every day before bed, and a homeopathic vaginal probiotic suppository every 3rd night before bed. In addition, for any vaginal insertion necessary (for manual therapy, dilation, or sex), women in the intervention group used only a specific personal iso-osmotic lubricant; and these women received a 4% lidocaine pain relief gel to be used as needed up to four times daily. The lubricant, gels, and wash were matched to healthy vaginal secretions for pH, osmolality, and lactic acid. At all study visits, all women completed questionnaires to self-report symptoms and were swabbed for vaginal microbiome and pH analysis. They also had vulvar skin quality and architecture assessed.

### EXAMPLE 12

The following describes a study to observe the effects of using a vaginal care system on the vaginal health in women undergoing physical therapy for hypertonic pelvic floor dysfunction. Premenopausal women between ages 18 and 52 referred to physical therapy for symptoms of hypertonic pelvic floor dysfunction were enrolled in a clinical study. After obtaining written informed consent, women were randomized to a control arm or an intervention arm, and were seen before starting the study to obtain the baseline and approximately weekly for therapy for 3 months. Women in the control group received routine care. Women in the intervention group received routine care and also used a vaginal care system consisting of three products: an external wash for intimate areas every day or as often as she bathes, an intravaginal moisturizing gel every other day before bed, and a homeopathic vaginal probiotic suppository every 3rd night before bed. In addition, for any vaginal insertion necessary (for manual therapy, use of vaginal dilators, or sex), women in the intervention group used only a specific personal iso-osmotic lubricant. The lubricant, gel, and wash were matched to healthy vaginal secretions for pH, osmolality, and lactic acid. At baseline, 6 wks, and 3 mo, all women completed questionnaires to self-report symptoms and were swabbed for vaginal microbiome and pH analysis. They also had pelvic floor muscle tone and other physical outcomes assessed. Data were analyzed to determine whether women who used the vaginal care system and iso-osmotic lubricant have faster or more complete resolution of symptoms of hypertonic pelvic floor disorder.

### EXAMPLE 13

The following describes a study to observe the effect of using a vaginal care system on recurrent bacterial vaginosis. Women were recruited for a 6-month observational clinical study. Between baseline and 6 months, women used a vaginal care system comprising three components: a daily feminine wash for external use, an intravaginal moisturizing gel used every other day before bed, and a homeopathic vaginal probiotic suppository used once every 3 days before bed. Women were tested at baseline, 3 months, and 6 months for vaginal microbiome by next-generation sequencing (NGS), vaginal pH, Nugent score, and presence of BV and yeast by the Affirm VPIII test.

Results: 12 women were recruited; 9 completed the study. Of the three methods used to assess presence of BV (NGS, Nugent score, and the Affirm VPIII test), there was good concordance between NGS and the Affirm VPIII test, and between NGS and the Nugent score, but poor concordance between the Affirm VPIII test and the Nugent score. Estimates show recurrence of BV in only one of nine patients (11%), lower than anticipated based on historic controls. No woman had a community state type IV (indicating BV) at the end of the study by NGS. Vaginal pH at the end of the study was 4.07±0.19 (mean±SD).

Conclusions/Implications: Results showed that the vaginal microbiome may be improved by regular use of a 3-part vaginal care system and that this system effectively reduced recurrence of BV.

### EXAMPLE 14

The following describes a study to observe the effect of using a vaginal care system on recurrent bacterial vaginosis. Women were recruited for a 6-month observational clinical study. Between baseline and 6 months, women used a vaginal care system comprising three components: a daily feminine wash for external use, an intravaginal moisturizing gel based on hydroxyethylcellulose used every other day before bed, and a homeopathic vaginal probiotic suppository used once every 3 days before bed. Women were tested at baseline, 3 months, and 6 months for vaginal microbiome by next-generation sequencing (NGS), vaginal pH, Nugent score, and presence of BV and yeast by the Aptima BV test.

Results: Microbiomes are dysbiotic at the start of the study in four out of five women by NGS, and vaginal pH was elevated (4.95±0.67). One woman corrected her microbiome within 12 weeks of use of the system.

Conclusions/Implications: The vaginal microbiome may be improved by regular use of a 3-part vaginal care system.

### EXAMPLE 15

The following describes a study to observe the effect of using a vaginal care system on recurrent symptomatic urinary tract infections. Premenopausal women between ages 18 and 52 diagnosed with recurrent symptomatic UTI were enrolled in a clinical study. After obtaining written informed consent, women were seen at baseline, 3 months, and 6 months. At all visits, all women completed a questionnaire to self-report symptoms and were swabbed for vaginal microbiome and pH analysis. Women used a vaginal care system consisting of three products: an external wash for intimate areas every day or as often as she bathes, an intravaginal moisturizing gel every other day before bed, and a homeopathic vaginal probiotic suppository every 3rd night before bed. The gel and wash were matched to healthy vaginal secretions for pH, osmolality, and lactic acid. Results were compared with historical controls.

### EXAMPLE 16

The following describes a study to observe the effect of using sexual lubricants on immune parameters. 215 women were recruited; approximately ⅓ had BV, ⅓ were healthy, and ⅓ had an intermediate vaginal microbiome. All participants had vaginal fluid sampled at baseline before and after a sexual arousal induction paradigm. Sexual arousal was assessed by measuring vaginal blood flow and autonomic arousal by electrocardiogram. Women completed surveys on sexual function and sexual health. Women then used a 3-part system at home for three weeks, consisting of a daily external feminine wash, intravaginal moisturizing gel every other day, and a homeopathic vaginal probiotic suppository every 3rd day. They completed daily diaries of vaginal symptoms and sexual arousal. After 3 weeks, women returned to the lab, repeating measurements obtained at baseline. Participants were randomized to hyperosmolar lubricant, iso-osmolar lubricant, or no lubricant; they were sent home with lubricants and a vibrator and asked to masturbate daily, rating symptoms daily. After 3 weeks, they returned for a final assessment of sexual function as before. Vaginal fluid samples at baseline, 3 weeks, and 6 weeks were assayed for pro- and anti-inflammatory cytokines and for microbial species presence and abundance.

Results: Results show that the majority of women (˜75%) had an intermediate vaginal microbiome at baseline; ˜20% had bacterial vaginosis (BV) and only ˜5% were healthy. After using the vaginal care system, ˜35% were healthy, ˜42% were intermediate, and ˜20% had BV.

Conclusions/Implications: The vaginal microbiome may be improved in as little as 3 weeks for many, but not all, women using the vaginal care system described. Better quality lubricants may improve sexual function and immune function.

### EXAMPLE 17

The following describes a study to observe the effect of using a vaginal care system on the vaginal microbiome and genitourinary symptoms in patients undergoing pelvic physical therapy for interstitial cystitis. Women between ages 18 and 89 referred to physical therapy for interstitial cystitis were enrolled in a clinical study. After obtaining written informed consent, women were randomized to a control arm or an intervention arm, and were seen at baseline, 3 months, and 6 months. Women in the control group received routine care. Women in the intervention group received routine care and used a vaginal care system consisting of three products: an external wash for intimate areas every day or as often as she bathes, an intravaginal moisturizing gel every other day before bed, and a homeopathic vaginal probiotic suppository every 3rd night before bed. In addition, for any vaginal insertion necessary (for manual therapy, dilation, or sex), women in the intervention group used only a specific personal iso-osmotic lubricant. The lubricant, gel, and wash were matched to healthy vaginal secretions for pH, osmolality, and lactic acid. At all study visits, all women completed questionnaires to self-report symptoms and were swabbed for vaginal microbiome and pH analysis. They also had pelvic floor muscle tone and other physical outcomes assessed. Data were analyzed to determine whether women who used the vaginal care system and iso-osmotic lubricant have faster or more complete resolution of symptoms of interstitial cystitis.

### EXAMPLE 18

The following describes a study to observe the effect of oral contraceptives and using a vaginal care system on the vaginal microbiome. Premenopausal women either using or not using combination oral contraceptive pills (OCP) were enrolled in this study. After obtaining written informed consent, women were randomized to a control arm or an intervention arm prior to delivery; women using OCP were randomized further to continue or discontinue use of OCP. Women in all groups were tested at baseline, 3 months, and six months for vaginal microbiome and pH. They were asked to complete a questionnaire on symptoms. All women received routine care for vaginal symptoms. Women who were randomized to the intervention group also used a vaginal care system consisting of three products: an external wash for intimate areas used every day or as often as she bathes, an intravaginal moisturizing gel every other day before bed, and a homeopathic vaginal probiotic suppository used every 3 days before bed. The gel and wash were matched to healthy vaginal secretions for pH, osmolality, and lactic acid. Vaginal microbiome composition, vaginal pH, and vulvovaginal self-reported symptoms at baseline, 3 months, and 6 months were assessed.

### EXAMPLE 19

The following describes a study to observe the effect of using a vaginal care system and fertility lubricant on conception. An open-label longitudinal randomized controlled trial enrolled 20 women in the intervention arm and 20 women in the control arm. All women gave written informed consent. Women were tested for vaginal microbiome and pH and completed an extensive questionnaire at baseline and 3 mo. Women in the intervention group used a vaginal care system: external wash daily, intravaginal gel every day before bed, and vaginal probiotic suppository every 3 days for 6 months. In addition, they used a fertility lubricant with low pH and low buffering capacity and calcium and magnesium ions during sex. Hydroxyethylcellulose-based gel and lubricant compositions matched healthy vaginal fluid for pH, osmolality, and lactic acid. Frequency of conception and pregnancy outcomes including gestational age at birth, complications, and rates of miscarriage/stillbirth were assessed.

### EXAMPLE 20

The following describes a study to observe the effect of using a vaginal care system on the vaginal microbiome and vulvovaginal symptoms in peripartum women. Women attending a birthing center for prenatal care and planning delivery with a certified nurse midwife (CNM) were invited to participate in this study. After obtaining written informed consent, women were randomized to either control arm or intervention arm. Upon onset of labor, women reported to the birthing center. During labor, prior to delivery, vaginal swabs were taken for baseline microbiome and pH analysis. All women were seen for follow-up at 2 wks and 6 wks; at 2 wks, vulvovaginal healing were assessed, and women were given a short vulvovaginal symptoms questionnaire. At 6 wks, healing assessment, questionnaire, and vaginal microbiome and pH testing were repeated. From delivery to 6 wks, women in the control arm received routine care and recommendations for vulvovaginal care at home. Women in the intervention arm were given a vaginal care system consisting of two products: an external wash for intimate areas to be used several times daily, and an intravaginal moisturizing gel every day before bed. In addition, they were given 4% lidocaine in a gel similar to the vaginal moisturizing gel to use as needed up to 4 times daily. The gels and wash were matched to healthy vaginal secretions for pH, osmolality, and lactic acid. Changes in vulvovaginal symptoms, vaginal microbiome, and vaginal pH over time, and degree of vulvovaginal healing were assessed at 2 wks and 6 wks.

### EXAMPLE 21

The following describes a study to compare the effects of bar soap and feminine wash on vaginal microbiome and recurrence of bacterial vaginosis. Premenopausal cis women ages 18-55 with history of recurrent BV who use bar soap for regular vulvar hygiene were invited to participate in the study. After obtaining written informed consent, vaginal swabs to test the microbiome were taken at baseline and at 3 months after switching from bar soap to a certain feminine wash. The wash was matched to healthy vaginal secretions for pH, osmolality, and lactic acid, and was used in place of soap while bathing. Presence of BV and yeast infection were assessed at baseline and 3 months; women who developed symptoms were optionally tested between those time points. Recurrence rates of BV and yeast infection by clinical molecular test, and self-report measures of vulvovaginal health were assessed by questionnaires. Outcomes were stratified by self-reported demographic information such as age, race, ethnicity, etc.

### EXAMPLE 22

The following describes a study to observe the effect of using vaginal care system on reduction of symptoms of endometriosis. Premenopausal women between ages 18 and 52 diagnosed with endometriosis and for whom vulvovaginal pain is a significant symptom were enrolled in a clinical study. After obtaining written informed consent, women were randomized to either an intervention arm or a control arm. All women were given routine care, including appointments at baseline, 3 months, and 6 months. At all visits, all women completed a questionnaire to self-report symptoms including vulvovaginal pain, other vulvovaginal symptoms such as dryness or irritation, pelvic pain, and urinary and digestive symptoms, and had vaginal swabs collected for microbiome and vaginal pH analysis. Women in the intervention group used a vaginal care system consisting of three products: an external wash for intimate areas every day or as often as she bathes, an intravaginal moisturizing gel every other day before bed, and a homeopathic vaginal probiotic suppository every 3rd night before bed. In addition, a 4% lidocaine gel made from a similar base as the vaginal moisturizing gel was offered for use as needed up to 4 times per day. The gels and wash were matched to healthy vaginal secretions for pH, osmolality, and lactic acid.

### EXAMPLE 23

The following describes a study to observe the effect of vaginal probiotics and pessaries and their impact on the vaginal microenvironment. Women ages 18-89 who use pessaries were enrolled; after obtaining informed consent, women were randomized to the experimental arm or standard care arm. Women in the experimental arm were given a vaginal moisturizing gel and a homeopathic vaginal probiotic suppository, with recommendation to use both of them 3 times per week before bed. The primary outcome was to examine changes in the vaginal microenvironment, including Lactobacilli, anaerobic bacteria, and Mobiluncus bacteria, white blood cells, and epithelial cell maturation. In addition, secondary outcomes were pelvic floor disability index; vaginal probiotic feasibility, compliance, and side effects of use; urinary tract infection rates; BVAB-1 bacteria abundance; levels of pro-inflammatory cytokines including interleukin (IL)-6, tumor necrosis factor (TNF)-alpha, IL-1alpha, IL-1beta; and incidence of adverse events.

### EXAMPLE 24

The following describes a study to observe the effect of feminine hygiene products on the vaginal microbiome and genitourinary syndrome of menopause in post-menopausal women with lower urinary tract symptoms. Postmenopausal women up to age 89 with lower urinary tract (LUT) symptoms of the genitourinary syndrome of menopause (GSM) were enrolled in the study. After obtaining written informed consent, women were examined at baseline and 3 months. At both visits, women completed a questionnaire to self-report on LUT and vulvovaginal symptoms and were swabbed for vaginal microbiome and pH analysis. Women used a vaginal care system consisting of three products: a wash every day or as often as she bathes, a gel every other day before bed, and a suppository every 3rd night before bed. The gel and wash were matched to healthy vaginal secretions for pH, osmolality, and lactic acid. The changes in LUT symptoms, vulvovaginal symptoms, vaginal microbiome, and vaginal pH before and after use of the vaginal care system were measured.

### EXAMPLE 25

The following describes a study to observe the effect of feminine hygiene products on recurrent urinary tract infection and vaginal health in post-menopausal women. Postmenopausal women up to age 89 diagnosed with recurrent symptomatic Urinary Tract Infection (UTI) were enrolled in a double-blind randomized controlled trial. After obtaining written informed consent, women were randomized to either control arm or intervention arm, and were examined at baseline, 3 months, and 6 months. At all visits, all women completed a questionnaire to self-report on vulvovaginal symptoms and were swabbed for vaginal microbiome, pH, and epithelial cell maturation index analysis. Women in the intervention group used a vaginal care system consisting of three products: a wash every day or as often as she bathes, a gel every other day before bed, and a suppository every 3rd night before bed. The gel and wash were matched to healthy vaginal secretions for pH, osmolality, and lactic acid. Women in the control group received similar competitor's products for wash and gel, and a placebo suppository. The frequency of UTI, yeast infection, and bacterial vaginosis, as well as changes in vulvovaginal symptoms, vaginal microbiome, vaginal epithelial maturation index, and vaginal pH over time were compared between intervention group and the control group.

### EXAMPLE 26

The following describes a study to observe the effect of feminine hygiene products on the vaginal microbiome in post-menopausal women using aromatase inhibitors (AIs). Postmenopausal women who take AIs for breast cancer therapy were enrolled in a clinical study. After obtaining written informed consent, women were randomized to either control arm or intervention arm and were examined at baseline and 3 months. At both visits, all women completed a questionnaire to self-report on vulvovaginal symptoms and were swabbed for vaginal microbiome, vaginal epithelial maturation index, and pH analysis. Women in the intervention arm used a vaginal care system consisting of three products: a wash every day or as often as she bathes, a gel every day before bed, and a suppository every 3rd day before bed. The gel and wash were matched to healthy vaginal secretions for pH, osmolality, and lactic acid. Women in the control arm did not use any specific vaginal care system, but continued using products they normally use. The changes in vulvovaginal symptoms, vaginal microbiome, vaginal epithelial maturation index, and vaginal pH over time were measured.

### EXAMPLE 27

The following describes a method for using a wipe, which may be combined with any of the Examples 7-26 above as an optional add-on procedure. A subject may use a wipe impregnated with the external wash solution for cleaning between showers, for example, between 6 days and 6 weeks, or from 6 weeks to 6 months. A kit containing feminine hygiene products may contain a wipe as a component or as an optional add-on item.

The disclosure set forth herein encompasses multiple distinct embodiments with independent utility. These embodiments are not to be considered in a limiting sense as numerous variations are possible. Each example defines an embodiment disclosed in the foregoing disclosure, but any one example does not necessarily encompass all features or combinations that may be eventually claimed. Where the description recites “a” or “a first” element or the equivalent thereof, such description includes one or more such elements, neither requiring nor excluding two or more such elements. Further, ordinal indicators, such as first, second or third, for identified elements are used to distinguish between the elements, and do not indicate a required or limited number of such elements, and do not indicate a particular position or order of such elements unless otherwise specifically stated.

### REFERENCES

1. Antunes AP, Schiefecker AJ, Beer R, et al. Higher brain extracellular potassium is associated with brain metabolic distress and poor outcome after aneurysmal subarachnoid hemorrhage. Crit Care. 2014;18(3):R119.
2. Sidorov VY, Woods MC, Wikswo JP. Effects of elevated extracellular potassium on the stimulation mechanism of diastolic cardiac tissue. Biophys J. 2003;84(5):3470-3479.
3. Parham WA, Mehdirad AA, Biermann KM, Fredman CS. Hyperkalemia revisited. Tex Heart Inst J. 2006;33(1):40-47.
4. Wagner G., Levin R.J. (1977) Human Vaginal Fluid, pH, Urea, Potassium and Potential Difference during Sexual Excitement. In: Gemme R., Wheeler C.C. (eds) Progress in Sexology. Perspectives in Sexuality (Behavior, Research, and Therapy). Springer, Boston, MA.
5. Mende HE, Spitzbart H, Sieke V, Vogel C. Natrium, Kalium, Magnesium and Calcium im Scheideninhalt [Sodium, potassium, magnesium and calcium in vaginal content]. Zentralbl Gynakol. 1990;112(18):1175-1180.
6. Wagner G, Levin RJ. Electrolytes in vaginal fluid during the menstrual cycle of coitally active and inactive women. J Reprod Fertil. 1980;60(1):17-27.
7. Owen, D.H. and Katz, D.F. (2005), A Review of the Physical and Chemical Properties of Human Semen and the Formulation of a Semen Simulant. Journal of Andrology, 26: 459-469.
8. Chooruk A, Utto P, Teanpaisan R, Piwat S, Chandeying N, Chandeying V. Prevalence of lactobacilli in normal women and women with bacterial vaginosis. J Med Assoc Thai. 2013;96(5):519-522.
9. Mirmonsef P, Gilbert D, Veazey RS, Wang J, Kendrick SR, Spear GT. A comparison of lower genital tract glycogen and lactic acid levels in women and macaques: implications for HIV and SIV susceptibility. AIDS Res Hum Retroviruses. 2012;28(1):76-81.
10. O'Hanlon DE, Moench TR, Cone RA. Vaginal pH and microbicidal lactic acid when lactobacilli dominate the microbiota. PLoS One. 2013;8(11):e80074.
11. O'Hanlon DE, Come RA, Moench TR. Vaginal pH measured in vivo: lactobacilli determine pH and lactic acid concentration. BMC Microbiol. 2019;19(1):13. Published 2019 Jan 14.
12. Beghini J, Linhares IM, Giraldo PC, Ledger WJ, Witkin SS. Differential expression of lactic acid isomers, extracellular matrix metalloproteinase inducer, and matrix metalloproteinase-8 in vaginal fluid from women with vaginal disorders. BJOG. 2015;122(12):1580-1585.
13. Binia A, Jaeger J, Hu Y, Singh A, Zimmermann D. Daily potassium intake and sodium-to-potassium ratio in the reduction of blood pressure: a meta-analysis of randomized controlled trials. J Hypertens. 2015 Aug;33(8):1509-20. doi: 10.1097/HJH.0000000000000611. PMID: 26039623.
14. Jayedi A, Ghomashi F, Zargar MS, Shab-Bidar S. Dietary sodium, sodium-to-potassium ratio, and risk of stroke: A systematic review and nonlinear dose-response meta-analysis. Clin Nutr. 2019 Jun;38(3):1092-1100. doi: 10.1016/j.clnu.2018.05.017. Epub 2018 Jun 1. PMID: 29907351.

1-23. (canceled)

24. A method for remediating a dysbiotic vaginal condition of a subject in need thereof, comprising:
applying an effective amount of a topical gel to a vagina of the subject;
applying a dosage of a suppository to the vagina of the subject; and
cleaning the vagina of the subject with an effective amount of a wash,
wherein the topical gel comprises:
water;
hydroxyethyl cellulose;
calcium chloride;
sodium chloride;
hyaluronic acid;
lactic acid; and
potassium chloride,
the suppository comprises:
homeopathic drugs; and
a probiotic blend comprising Lactobacillus acidophilus LA02 (DSM 21717), Lactobacillus crispatus LCR01 (DSM 24619), Lactobacillus gasseri LGS06 (DSM 32405), Limosilactobacillus fermentum LF08 (DSM 18297), Lactiplantibacillus plantarum LP01 (LMG P-21021), Lacticaseibacillus rhamnosus LR06 (DSM 21981), and Ligilactobacillus salivarius CRL1328 (DSM 24441), and
the wash comprises:
water;
cocamidopropyl hydroxysultaine; and
lactic acid,
wherein the applying the topical gel, the applying the suppository, and the cleaning the vagina are performed regularly.

25. The method according to claim 24,
wherein the wash further comprises Aloe barbadensis leaf juice.

26. The method according to claim 24,
wherein the applying the topical gel, applying the suppository, and cleaning the vagina of the subject is performed regularly until the dysbiotic vaginal condition of the subject is remediated.

27. The method according to claim 26,
wherein the method is continued after the dysbiotic vaginal condition has been remediated.

28. The method according to claim 24,
wherein the subject previously had a compromised vaginal condition, and the method is performed after the compromised vaginal condition has been resolved.

29. The method according to claim 24,
wherein applying the topical gel is performed more than once every day.

30. The method according to claim 24,
wherein applying the topical gel is performed once every day or less frequently.

31. The method according to claim 24,
wherein the cleaning the vagina of the subject is performed more than once every day.

32. The method according to claim 24,
wherein the cleaning the vagina of the subject is performed once every day or less frequently.

33. The method according to claim 24,
wherein the suppository is applied once every day or less frequently.

34. The method according to claim 24,
wherein the subject treated with the method has lower vaginal pH as soon as twelve weeks after starting the method compared with the vaginal pH before starting of the method.

35. The method according to claim 24,
wherein the subject treated with the method has lower chance of having a recurrence of the dysbiotic vaginal condition compared with a subject not treated with the method.

36. The method according to claim 24, wherein
the topical gel comprises, in percentage by weight, water at 94.5-97.5%, hydroxyethyl cellulose at 0.1-5.0%, calcium chloride at 0.1-1.5%, sodium chloride at 0.1-1.5%, hyaluronic acid at 0.1-4.0%, lactic acid at 0.1-4.0%, and potassium chloride at 0.01-1.5%,
the suppository comprises, in percentage by weight, homeopathic drugs at 0.1-5.0% and the probiotic blend at 25.0-50.0%, and
the wash comprises, in percentage by weight, water at 60.0-70.0%, cocamidopropyl hydroxysultaine at 3.0-20.0%, lactic acid at 0.25-4.0%.

37. The method according to claim 25, wherein the percentage by weight of the Aloe barbadensis leaf juice is 0.1-5.0%.

38. The method according to claim 24,
wherein the dysbiotic condition is caused by at least one medical condition selected from the group consisting of bacterial vaginosis, recurrent urinary tract infection, recurrent candidiasis, aerobic vaginitis, desquamative inflammatory vaginitis, endometriosis, adenomyosis, lactation-induced vaginal condition, a treatment for a hormone-sensitive cancer, a medically- or disease-induced low-estrogen state, including a use of oral contraceptives, an oophorectomy, adrenal dysfunction, and pituitary or ovarian failure, stress, male-to-female gender transition after gender confirmation surgery, female-to-male gender transition prior to gender confirmation surgery or any similar low estrogen, high testosterone conditions, polycystic ovary syndrome (PCOS), interstitial cystitis, an use of oral contraceptives, sexual trauma, lichen sclerosus, lichen planus, delivery, and combinations thereof.

39. The method according to claim 28,
wherein the compromised vaginal condition was caused by at least one medical condition selected from the group consisting of vulvodynia, vestibulodynia, localized provoked vulvodynia, a vaginal surgery, a neovaginal construction, a transvaginal hysterectomy, a pelvic radiation and/or a brachytherapy, a treatment for a hormone-sensitive cancer, a medically- or disease-induced low-estrogen state, including a use of oral contraceptives, an oophorectomy, adrenal dysfunction, and pituitary or ovarian failure, lichen sclerosus and/or lichen planus, endometriosis, genital graft-vs-host disease, pelvic inflammatory disease, hypertonic or hypotonic pelvic floor disorders, Sjögren syndrome, dermatitis, delivery, and combinations thereof.
